SEQUENCE LISTING (3 sequences)

SEQ ID NO: 1 - protein, 135 aa, Human
CQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKCPFADQALF

SEQ ID NO: 2 - protein, 129 aa, Human
CQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKVRL

SEQ ID NO: 3 - protein, 129 aa, Cynomolgus
CQGSKYSTCASCPSCPDHWMKYGNHCYYFSVEKKDWISSLEFCLARDSHLLMITDKQEMSLLQDFLSEAFHWVGLRNNSGWRWEDGSPLNFSRIYSNSLVQTCGAINKNSLQASSCEVSLQWVCKKVRL

(19) United States
(12) Patent Application Publication
(10) Pub. No.: US 2019/0194333 A1
(43) Pub. Date: Jun. 27, 2019

(54) KLRG1 DEPLETION THERAPY

(71) Applicants: THE BRIGHAM AND WOMEN'S HOSPITAL, INC., Boston, MA (US); CHILDREN'S MEDICAL CENTER CORPORATION, Boston, MA (US)

(72) Inventors: Steven Greenberg, Watertown, MA (US); Stefano Vincenzo Gulla, Medford, MA (US); Kenneth Evan Thompson, Arlington, MA (US)

(21) Appl. No.: 16/327,576

(22) PCT Filed: Sep. 15, 2017
(86) PCT No.: PCT/US17/51776

§ 371 Date: Feb. 22, 2019

Related U.S. Application Data
(60) Provisional application No. 62/395,551, filed on Sep. 16, 2016.

(51) Int. Cl. C07K 16/28; A61P 37/08; A61P 37/06; A61P 35/00; A61K 47/68; C07K 14/705

(52) U.S. Cl. CPC C07K 16/2851; A61P 37/08; A61P 37/06; A61P 35/00; A61K 38/00; C07K 14/705; C07K 16/2818; C07K 16/2827; A61K 47/6801

(57) ABSTRACT

A method of treating a subject can comprise administering to a subject in need thereof an effective amount of a killer cell lectin-like receptor G1 (KLRG1) depleting agent, thereby depleting CD8+ cytotoxic T and/or NK cells in vivo. A method of treating a subject can comprise administering to a subject in need thereof an effective amount of a KLRG1 depleting agent with effector killing function. A composition, for example an anti-KLRG1 antibody, can comprise a KLRG1 depleting agent. The methods and compositions can be used for various diseases in which KLRG1 is overexpressed, for example autoimmune diseases, transplant rejection, hematologic malignancies, and solid tumors.

The present application is a national stage application of International Application No. PCT/US2017/051776, filed Sep. 15, 2017, which claims priority to and the benefit of U.S. Provisional Application Ser. No. 62/395,551, filed on Sep. 16, 2016, and titled, “KLRG1 DEPLETION THERAPY,” the contents of each which is incorporated herein by reference in their entireties.

### FIELD OF THE INVENTION

The present invention generally relates to KLRG1-expressing-cell depletion therapies and therapeutics. In various embodiments, the present invention more specifically relates to KLRG1-expressing-cell depletion therapies and therapeutics for autoimmune disease, transplant rejection, hematologic malignancies, and solid tumors.

### BACKGROUND

Cellular injury occurs in many diseases as a consequence of cytotoxic T cell attack. For example, pathogenic cytotoxic T cells are a key element in the destruction of muscle that occurs in the disease inclusion body myositis (Arahata and Engel, 1984, Arahata and Engel, 1986, Arahata and Engel, 1988, Amemiya et al., 2000). Similar mechanisms of injury to tissues by cytotoxic T cells are implicated in other autoimmune diseases (Blanco et al., 2005) such as multiple sclerosis (Zang et al., 2004, Friese and Fugger, 2009), rheumatoid arthritis (Carvalheiro et al., 2014), psoriasis (Hijnen et al., 2013), inflammatory bowel disease (Muller et al., 1998, Bisping et al., 2001), autoimmune thyroid disease (Okajima et al., 2009), type 1 diabetes (Faustman and Davis, 2009), alopecia areata (Xing et al., 2014), Bechet's disease (Yu et al., 2004), ankylosing spondylitis (Schirmer et al., 2002, Trevino et al., 2004), and primary biliary cirrhosis (Kita, 2007). Similar mechanisms of injury are also present in solid organ transplantation, such as in graft versus host disease and organ rejection developing in the setting of transplantation associated with attack on tissues by CD8+ T cells (Bueno and Pestana, 2002), where there are increased proportions of highly potent increased differentiated T cells, such as T effector memory (TEM) and T effector memory RA (TEMRA) (D'Asaro et al., 2006, Betjes et al., 2012). Additionally, certain leukemias and lymphomas also involve the abnormal expansion of CD8+ T cells. In particular, T cell large granular lymphocytic leukemia (T-LGLL) is a leukemia characterized by expansion of late-stage differentiated CD8+ T cells, and NK cell lymphoproliferative disorder is a leukemia characrerized by NK cell expansion (Lamy and Loughran, 2011). Extranasal NK/T cell lymphoma is a related disorder (Takata et al., 2015). Inclusion body myositis may overlap substantially with T cell large granular lymphocytic leukemia (T-LGLL). In one series, 58% of patients with IBM met published diagnostic criteria for T-LGLL (Greenberg et al., 2016). Numerous cell surface molecules expressed by cytotoxic T cells have been identified. However, therapeutic developments based upon targeting these molecules are limited and there remains a need for new therapeutics utilizing these targets for various indications including autoimmune disease, transplant rejection, hematologic malignancies, and solid tumors.

### SUMMARY OF THE INVENTION

The invention is based, at least in part, on the discovery that killer cell lectin-like receptor G1 (KLRG1), a cell surface marker known to be present on senescent cytotoxic T cells, is also present on cytotoxic T cells with high-killing potential. For example, in the case of inclusion body myositis, KLRG1 marks T cells that are directly killing healthy muscle cells. Unlike the teachings of prior studies regarding the senescent and inactive nature of KLRG1-expressing T cells in the blood of mice and humans, KLRG1-expressing T cells can be pathogenic and are therefore an advantageous target for cell depletion therapy. For example, administering to a subject in need thereof an effective amount of KLRG1 depleting agent (e.g., a KLRG1-expressing-cell depleting agent) with antibody dependent cellular cytotoxicity (ADCC) effector function can eliminate or reduce the number of cytotoxic T cells and/or NK cells injuring healthy cells.

Thus, the invention has numerous therapeutic uses. For example, the invention can be used for treating inclusion body myositis (IBM). More generally, the invention can be used for treating, including in some cases preventing, various diseases associated with KLRG1-expressing cells (i.e., by depleting the KLRG1-expressing cells). Embodiments of the invention include treating, and in some cases preventing, autoimmune diseases, transplant rejection, hematologic malignancies, and solid tumors.

Advantages of the invention include the ability to preferentially target CD8+ cytotoxic T and/or NK cells for depletion and potentially greater efficacy and reduced side effects. The population of KLRG1-expressing immune cells more abundantly express cytotoxic molecules than the population of total CD2+ or CD3+ expressing T cells, and are more specific to cytotoxic T cells than CD52+ hence potential for greater efficacy. KLRG1 is a marker that increases with antigen experience, predicting more specific antigen directed immune responses and, hence potential for greater efficacy and reduced side effects.

In various aspects, the invention provides a method of treating a subject comprising administering to a subject in need thereof an effective amount of a killer cell lectin-like receptor G1 (KLRG1) depleting agent (a KLRG1-expressing-cell depleting agent), thereby depleting CD8+ cytotoxic T and/or NK cells in vivo. The KLRG1 depleting agent can specifically target and deplete CD8+ cytotoxic T and/or NK cells expressing KLRG1.

In various aspects, the invention provides a method of treating a subject comprising administering to a subject in need thereof an effective amount of a killer cell lectin-like receptor G1 (KLRG1) depleting agent (a KLRG1-expressing-cell depleting agent) with effector killing function. The KLRG1 depleting agent can specifically target and deplete pathogenic, or otherwise harmful or undesired, cells expressing KLRG1.

In various aspects, the invention uses a killer cell lectin-like receptor G1 (KLRG1) depleting agent (a KLRG1-expressing-cell depleting agent).

In various aspects, the invention uses an mRNA or cDNA encoding the depleting agent.

In various aspects, the invention uses a pharmaceutical composition comprising an effective amount of the depleting agent.

As will be understood by those skilled in the art, any of the aspects above can be combined with any one or more of the features below.

In various embodiments, the depleting agent is an antibody or antigen binding fragment thereof, or antibody mimetic.

In various embodiments, the antibody is monoclonal.

In various embodiments, the antibody or antigen binding fragment thereof, or antibody mimetic comprises a human or humanized antibody.

In various embodiments, the antibody or antigen binding fragment thereof, or antibody mimetic comprises: a. a full length antibody Fab antibody that binds KLRG1 with effector function antibody dependent cell-mediated cytotoxicity (ADCC); b. an antibody that binds KLRG1 with effector function complement dependent cytotoxicity (CDC); c. an antibody that binds KLRG1 with effector function antibody-drug conjugate (ADC); d. an Fc-cadherin fusion protein; e. a fusion protein E-cadherin/Fc; f. a fusion protein R-cadherin/Fc; g. a fusion protein N-cadherin/Fc; h. a chimeric antigen receptor; or i. a multispecific antibody.

In various embodiments, the chimeric antigen receptor and wherein the chimeric antigen receptor comprises a specificity portion of a KLRG1 antibody grafted onto a T cell.

In various embodiments, the multispecific antibody comprises a bispecific or trispecific antibody.

In various embodiments, the depleting agent binds KLRG1.

In various embodiments, the KLRG1 is the extracellular domain of human KLRG1.

In various embodiments, the depleting agent cross reacts with the extracellular domains of human and cynomolgus KLRG1.

In various embodiments, the depleting agent binds to an epitope of the extracellular domain of KLRG1, wherein the epitope is at least 90% identical in human and cynomolgus.

In certain embodiments, the depleting agent binds to KLRG1 and is not clone 13F12F2, 14C2A07, REA261, 13A2, SA231A2, 2F1, 13A2, or REA261.

In certain aspects, the invention uses a killer cell lectin-like receptor G1 (KLRG1) depleting agent, other than any of the agents described in PCT Application No. PCT/US17/35621.

In certain embodiments, the depleting agent binds to KLRG1 and is not a mouse antibody.

In various embodiments, the depleting agent binds KLRG1, thereby labeling CD8+ cytotoxic T and/or NK cells for depletion.

In various embodiments, the depleting agent binds KLRG1, thereby inducing Antibody-Dependent Cellular Cytotoxicity (ADCC) or Complement Dependent Cytotoxicity (CDC).

In various embodiments, the depleting agent selectively targets and depletes T and/or NK cells expressing KLRG1.

In various embodiments, the depleting agent is administered by providing an mRNA encoding the depleting agent to the subject.

In various embodiments, the subject has an autoimmune disease.

In various embodiments, the autoimmune disease is rheumatoid arthritis, psoriasis, inclusion body myositis (IBM), multiple sclerosis, ulcerative colitis, lymphocytic colitis, idiopathic thrombocytopenic purpura, primary biliary cholangitis, or type 1 diabetes.

In various embodiments, the subject has or is at risk of developing transplant rejection.

In various embodiments, the transplant rejection is kidney rejection, preferably T cell mediated kidney rejection after transplantation.

In various embodiments, the subject has a hematologic malignancy.

In various embodiments, the hematologic malignancy is a leukemia.

In various embodiments, the leukemia is T cell leukemia, NK cell leukemia, large granular lymphocytic leukemia (LGLL), or chronic lymphocytic leukemia (CLL).

In various embodiments, the subject has a lymphoma.

In various embodiments, the lymphoma is T cell lymphoma, preferably anaplastic large cell lymphoma.

In various embodiments, the subject has a solid tumor.

In various embodiments, the solid tumor is a breast cancer, gastric cancer, ovarian cancer, prostate cancer, glioma, glioblastoma, melanoma, lung cancer, kidney cancer, or tongue cancer. The lung cancer can be, for example, non-small cell lung cancer. The kidney cancer can be, for example, renal cell carcinoma.

In various embodiments, the method of treatment further comprises administering to the subject an effective amount of a checkpoint modulator therapy.

In various embodiments, the KLRG1 depleting agent and checkpoint modulator therapies are synergistic.

In various embodiments, the checkpoint modulator therapy comprises an anti-PD-1, anti-PD-L1, or anti-CTLA-4 therapy.

In various embodiments, the subject has failed or has not responded to a prior cancer therapy.

In various embodiments, the invention uses the KLRG1 depleting agent in the preparation of medicament for treatment or prevention of an autoimmune disease, a transplant rejection, a hematologic malignancy, or a solid tumor.

These and other advantages of the present technology will be apparent when reference is made to the accompanying drawings and the following description.

### BRIEF DESCRIPTION OF THE DRAWINGS

FIG. 1 shows that human KLRG1 is expressed on greater proportions of cytotoxic T and NK cells than helper T cells.

FIGS. 2A and 2B show a progression of increasing expression of KLRG1 on T cells with increased differentiation.

FIG. 3 shows increased KLRG1 gene expression in IBM muscle compared to normal muscle. Twelve IBM muscle biopsies compared with 5 normal muscle biopsies.

FIG. 4 shows expression of KLRG1 by immunohistochemistry on muscle-invading T cells in inclusion body myositis (4 patient samples shown).

FIG. 5 shows expression of KLRG1 in lymph node, demonstrating the vast majority of CD8+ T cells in lymph node do not express KLRG1.

FIGS. 6A and 6B show sera response of immunized mice to KLRG1.

FIG. 7 shows binding of antibodies derived from hybridoma clones to human KLRG1 extracellular domain.

FIGS. 8A-8C show KLRG1+ T cells in blood are increased in abundance in patients with IBM compared to age-matched healthy individuals.

FIGS. 9A and 9B show expression of KLRG1 on CD8+CD57+ blood T cells in a patient with inclusion body myositis and large granular lymphocytic leukemia.

FIGS. 10A and 10B show expression of KLRG1 by immunohistochemistry on tissue-invading leukemic cells in large granular lymphocytic leukemia (LGLL) and chronic lymphocytic leukemia (CLL), respectively.

FIG. 11 shows that KLRG1 is slightly overexpressed in intestinal biopsies from patients with ulcerative colitis.

FIG. 12 shows that KLRG1 is overexpressed in patients with idiopathic thrombocytopenic purpura.

FIG. 13 shows that KLRG1 is overexpressed in colon biopsies from patients with lymphocytic colitis.

FIG. 14 shows that KLRG1 is overexpressed in kidney biopsies from patients with T cell mediated kidney rejection after transplantation.

FIG. 15 shows that KLRG1 is overexpressed in lymph node from patients with anaplastic large cell lymphoma compared with normal CD4+ T cells.

FIG. 16 shows that KLRG1 is overexpressed in synovial biopsies from patients with rheumatoid arthritis.

FIG. 17 shows that KLRG1 is overexpressed in skin biopsies from patients with psoriasis.

FIG. 18 shows that KLRG1 is overexpressed in liver biopsies from patients with primary biliary cholangitis.

FIG. 19 shows that KLRG1 is overexpressed in pancreas tissue from patients with type 1 diabetes.

FIG. 20 shows that KLRG1 is overexpressed in blood from patients with T-cell large granular lymphocytic leukemia.

FIG. 21 shows that KLRG1 is expressed by T cells in T cell leukemias and lymphomas.

FIG. 22 shows that KLRG1 is slightly overexpressed in brain from patients with multiple sclerosis.

FIG. 23 shows widespread infiltration of many tumor types by KLRG1-expressing T cells.

FIGS. 24A-24C show KLRG1+ cells infiltrating melanoma tumors in 3 patients.

FIG. 24D shows no KLRG1+ cells in normal skin.

FIG. 25 shows KLRG1+ cells infiltrating renal cell carcinoma tumors in 4 patients.

FIG. 26 shows KLRG1+ cells infiltrating non-small cell lung cancer tumors in 4 patients.

FIGS. 27A-27C show depletion of CD8+CD57+ terminally differentiated cells using a KLGR1 depleting agent.

FIG. 28 shows that KLRG1 is overexpressed in tongue biopsies from patients with tongue carcinoma.

While the invention comprises embodiments in many different forms, there are shown in the drawings and will herein be described in detail several specific embodiments with the understanding that the present disclosure is to be considered as an exemplification of the principles of the technology and is not intended to limit the invention to the embodiments illustrated.

### BRIEF DESCRIPTION OF THE SEQUENCES

SEQ ID NO:1 is the sequence of human KLRG1 extra cellular domain (ECD) isotype 1.

SEQ ID NO:2 is the sequence of human KLRG1 ECD isotype 2.

SEQ ID NO:3 is the sequence of cynomolgus KLRG1 ECD.

### DETAILED DESCRIPTION

The invention is based, at least in part, on the discovery that KLRG1, a cell surface marker known to be present on senescent cytotoxic T cells, is also present on cytotoxic T cells with high-killing potential. In the case of inclusion body myositis, KLRG1 marks T cells that are directly killing human muscle cells. Unlike the teachings of prior studies regarding the senescent and inactive nature of KLRG1-expressing T cells in the blood of mice and humans, KLRG1-expressing T cells in certain samples are pathogenic and are therefore a favorable target for depletion therapy. For example, administering to a subject in need thereof an effective amount of killer cell lectin-like receptor G1 (KLRG1) depleting agent with antibody dependent cellular cytotoxicity (ADCC) effector function can eliminate or reduce the number of cytotoxic T cells injuring cells. Thus, the invention has numerous therapeutic uses, particularly in diseases where a subset of pathogenic cells overexpressing KLRG1, e.g., 1.2-fold, 1.3-fold, 1.4-fold, 1.5-fold, 2-fold, 3-fold, 4-fold, 5-fold, 10-fold, 20-fold, 30-fold, 40-fold, 50-fold, 60-fold, 70-fold, 80-fold, 90-fold, 100-fold and/or higher, relative to healthy subjects. The subset of pathogenic cells may be, for example, CD8+ cytotoxic T cells or NK cells. For example, the invention can be used for treating inclusion body myositis. More generally, the invention can be used for treating, and in some cases preventing, autoimmune disease, transplant rejection, hematologic malignancies, and solid tumors.

Advantages of the invention include the ability to preferentially target CD8+ cytotoxic T and/or NK cells for depletion. Advantages of the invention also include greater efficacy and reduced side effects. For example, the population of KLRG1-expressing immune cells more abundantly express cytotoxic molecules than the population of total CD2+ or CD3+ expressing T cells, and are more specific to cytotoxic T cells than CD52 hence potential for greater efficacy; and KLRG1 is a marker that increases with antigen experience, predicting more specific antigen directed immune responses and, hence potential for greater efficacy and reduced side effects.

In various aspects, the invention provides a method of treating a subject comprising administering to a subject in need thereof an effective amount of a killer cell lectin-like receptor G1 (KLRG1) depleting agent, thereby depleting CD8+ cytotoxic T and/or NK cells in vivo. The treatment can be for an autoimmune disease, transplant rejection, hematologic malignancies, and solid tumors (examples discussed below).

In various aspects, the invention also provides a method of treating a subject comprising administering to a subject in need thereof an effective amount of a killer cell lectin-like receptor G1 (KLRG1) depleting agent with effector killing function. Again, the treatment can be for an autoimmune disease, transplant rejection, hematologic malignancies, and solid tumors (examples discussed below).

In various aspects, the invention uses a killer cell lectin-like receptor G1 (KLRG1) depleting agent. In various aspects, the invention uses an mRNA or cDNA encoding the depleting agent. In various aspects, the invention uses a pharmaceutical composition comprising an effective amount of the depleting agent.

Various features of the invention, including KLRG1 and its ligands, depleting agents, pharmaceutical compositions, treatment and administration, and illustrative examples are discussed, in turn, below.

Killer Cell Lectin-Like Receptor G1 (KLRG1) and its Ligands

Killer cell lectin-like receptor G1 (KLRG1) is type II transmembrane protein and is a co-inhibitory receptor modulating the activity of T and NK cells. Its extracellular portion contains a C-type lectin domain whose known ligands are cadherins and its intracellular portion contains an immunoreceptor tyrosine-based inhibitory motif (ITIM) domain responsible for co-inhibition of T cell receptor (TCR) mediated signaling (Tessmer et al., 2007). In various embodiments, the ligand can be E-cadherin, N-cadherin, R-cadherin, or a combination thereof.

KLRG1 distribution and function differ in the rodent compared to the human. Originally identified on a rodent mast cell line (Guthmann et al., 1995), KLRG1 is not expressed on human mast cells, basophils, monocytes, or neutrophils (Voehringer et al., 2002). Human KLRG1 is a more potent co-inhibitory receptor than mouse KLRG1. KLRG1-mediated inhibition under physiological conditions is only observed with human lymphocytes because KLRG1 dimers have greater potency than monomers, and human KLRG1 forms exclusively dimers while mouse KLRG1 exists as monomers and dimers (Hofmann et al., 2012).

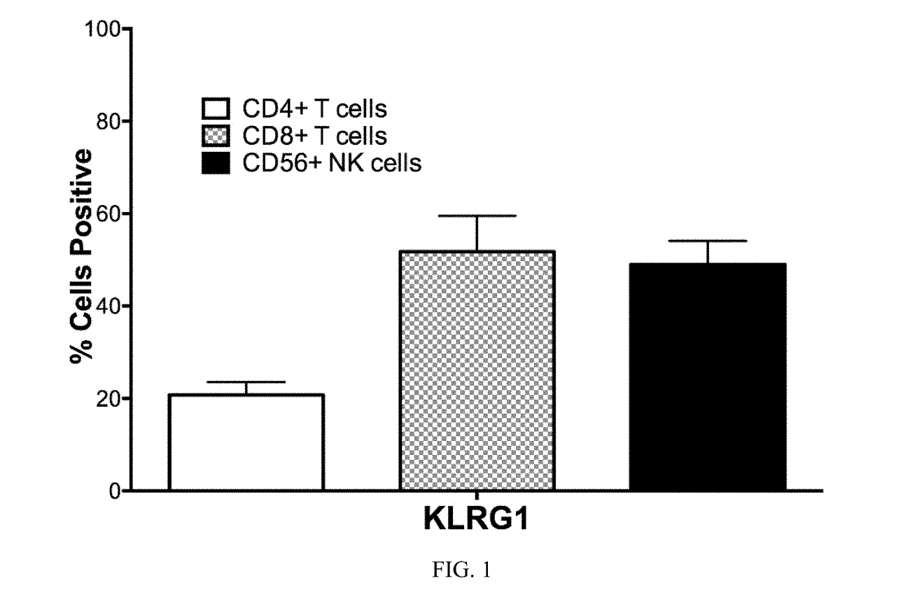

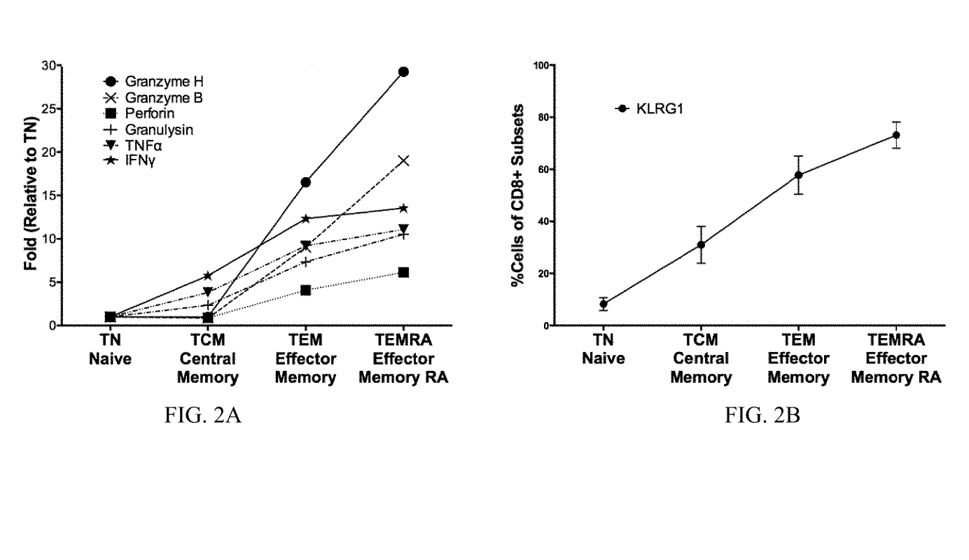

KLRG1 expression in humans is limited to T and NK cells. It is expressed on greater proportions of cytotoxic T and NK cells than helper T cells (FIG. 1, KLRG1 expression of lymphocyte subsets, human blood flow cytometry). Specifically, FIG. 1 shows the greater expression of KLRG1 on cytotoxic CD8+ T cells and NK cells than on CD4+ helper T cells. Within the CD8+ cytotoxic T cell population, increased KLRG1 expression correlates with increased antigen specific potency, as shown in FIGS. 2A and 2B. Specifically, FIGS. 2A and 2B show increasing expression of KLRG1 on T cells with increased differentiation. FIG. 2A (cytotoxicity and cytokines of CD8+ T cell subsets, human blood gene expression) shows that cytotoxic potential of T cells increases from TN→TCM→TEM→TEMRA. FIG. 2B (% KLRG1+CD6+ T cells of human blood CD8+ subpopulation by flow cytometry) shows that KLRG1 expression increases from TN→TCM→TEM→TEMRA. As CD8+ cytotoxic T cells differentiate in response to antigen, from naïve T cells to central memory, effector memory, and effector memory RA cells, they express increased amounts of KLRG1. Thus, KLRG1 marks cells with high capacity for cytotoxic killing. This cytotoxicity may be undesired (in the case of autoimmune disease and transplant rejection) or desired (in the case of cancer or chronic infectious disease).

As KLRG1 function in humans is substantially different than KLRG1 function in mice, mouse data is of limited applicability to the treatment of human disease. However, there is a near complete absence of published KLRG1 translational data in human diseases. There are no published studies of KLRG1 expression by immunohistochemistry in any human diseased or healthy tissue sample. There are 4 published studies that contain minor data on KLRG1 expression by flow cytometry in human diseased tissue samples, other than peripheral blood mononuclear cells (PBMCs): tumor-infiltrating lymphocytes in hepatocellular carcinoma (Brunner et al., 2015) and renal cell carcinoma (Attig et al., 2009); tumor infiltrated lymph node in melanoma (Legat et al., 2013); and synovial T cells in rheumatoid arthritis and spondyloarthropathies (Melis et al., 2014). Approximately 5 published studies contain minor data on KLRG1 expression in diseased PBMCs. (See e.g., references in Example 2.)

KLRG1 is a marker of immunosenescence (Akbar and Henson, 2011, Apetoh et al., 2015).

In various aspects and embodiments, KLRG1 is human or cynomolgus KLRG1, preferably human KLRG1, including any functional part thereof. For example, KLRG1 can be Human-KLRG1-ECD-Isotype 1 (SEQ ID NO:1), including any functional part thereof.

In various aspects and embodiments, KLRG1 is Human-KLRG1-ECD-Isotype2 (SEQ ID NO:2), including any functional part thereof.

In various aspects and embodiments, KLRG1 is Cynomolgus-KLRG1 (SEQ ID NO: 3), including any functional part thereof.

Depleting Agents

In various aspects and embodiments, the invention provides a method of treating a subject comprising administering to a subject in need thereof an effective amount of a killer cell lectin-like receptor G1 (KLRG1) depleting agent. As used herein, the term “depleting agent” is an agent that substantially reduces the number of a specific cell population. The cell population targeted by the depleting agent is identified by at least one characteristic feature, for example a cell surface marker (e.g., the presence and/or overexpression of KLRG1 relative to other cells). In the case of a “KLRG1 depleting agent,” the agent reduces the number of KLRG1-expressing and/or overexpressing cells (e.g., the KLRG1 depleting agent does not deplete KLRG1 in isolation, but rather depletes cells characterized by KLRG1).

In various embodiments, the depleting agent used in the methods of the invention is capable of reducing the number of the targeted cell population by 5%, 10%, 20%, 30%, 40%, 50%, 60%, 70%, 80%, 90%, 95%, 99%, and/or 100% relative to untreated target cells or target cells treated with IgG1 isotype antibody.

In various embodiments, the depleting agent used in the methods of the invention is an antibody or antigen binding fragment thereof, or antibody mimetic with effector function antibody dependent cell-mediated cytotoxicity (ADCC), effector function complement dependent cytotoxicity (CDC); effector function antibody-drug conjugate (ADC); a fusion protein that binds specifically to the targeted cell type (e.g., a ligand of a receptor specific to the targeted cell type) and effects cell killing via conjugation to a drug or an immunoglobulin with effector ADCC or CDC function; or a small molecule agent that specifically targets (e.g., binds) and depletes a cell type (e.g., by inducing cell death or destruction, for example via toxin delivery or metabolic alterations).

In various embodiments, the depleting agent is an antibody or antigen binding fragment thereof, or antibody mimetic. The term “antibody” is used in the broadest sense and covers, for example, single anti-KLRG1 monoclonal antibodies. The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody.

“Antibody fragments” can include a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (C_{H}1) of the heavy chain. Fab fragments differ from Fab′ fragments by the addition of a few residues at the carboxy terminus of the heavy chain C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. “Single-chain Fv” or “sFv” antibody fragments comprise the V_{H} and V_{L} domains of antibody, wherein these domains are present in a single polypeptide chain. Preferably, the Fv polypeptide further comprises a polypeptide linker between the V_{H} and V_{L} domains which enables the sFv to form the desired structure for antigen binding.

In various embodiments, the antibody or antigen binding fragment thereof, or antibody mimetic comprises a human or humanized antibody. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The depleting agents may also be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity matured antibody has nanomolar or even picomolar affinities for the target antigen. Preferred affinity matured antibodies have an affinity which is five times, more preferably 10 times, even more preferably 20 or 30 times greater than the starting antibody (generally murine, humanized or human) from which the matured antibody is prepared.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. As such, a KLRG1 depleting agent includes functional equivalents to an anti-KLRG1 antibody according to the invention. A KLRG1 depleting agent can be a binding agent that binds to or specifically binds to KLRG1 (e.g., human KLRG1), for example native KLRG1 on a cell surface. In some cases, the KLRG1 binding agent may be cross reactive with various similar KLRG1 proteins (e.g., with highest affinity for one, such as human KLRG1, and lower affinity for others, such as mouse KLRG1).

In various embodiments, the depleting agent is a blocking or antagonist binding agent. “Blocking” or “antagonist” means the agent (e.g., antibody or binding fragment/mimic thereof) is one which inhibits or reduces biological activity of the antigen it binds. Certain blocking agents or antagonist agents substantially or completely inhibit the biological activity of the antigen. For example, a KLRG1 binding agent can block KLRG1 signaling (e.g., thereby disrupting KLRG1 signaling and activating CD8+ cytotoxic T and/or NK cells).

In various embodiments, the antibody or antigen binding fragment thereof, or antibody mimetic comprises: a. a full length antibody Fab antibody that binds KLRG1 with effector function antibody dependent cell-mediated cytotoxicity (ADCC); b. an antibody that binds KLRG1 with effector function complement dependent cytotoxicity (CDC); c. an antibody that binds KLRG1 with effector function antibody-drug conjugate (ADC); d. an Fc-cadherin fusion protein; e. a fusion protein E-cadherin/Fc; f. a fusion protein R-cadherin/Fc; g. a fusion protein N-cadherin/Fc; h. a chimeric antigen receptor; or i. a multispecific antibody.

In various embodiments, the chimeric antigen receptor comprises a specificity portion of a KLRG1 antibody grafted onto a T cell.

In various embodiments, the multispecific antibody comprises a bispecific or trispecific antibody.

In various embodiments, the depleting agent binds KLRG1.

In various embodiments, the KLRG1 is the extracellular domain of human KLRG1.

In various embodiments, the depleting agent cross reacts with the extracellular domains of human and cynomolgus KLRG1.

In various embodiments, the depleting agent binds to an epitope of the extracellular domain of KLRG1, wherein the epitope is at least 90% identical in human and cynomolgus.

In various embodiments, the depleting agent binds to KLRG1 and is not a mouse antibody.

In various embodiments, the depleting agent binds KLRG1, thereby labeling CD8+ cytotoxic T and/or NK cells for depletion.

In various embodiments, the depleting agent binds KLRG1, thereby inducing Antibody-Dependent Cellular Cytotoxicity (ADCC) or Complement Dependent Cytotoxicity (CDC).

In various embodiments, the depleting agent selectively targets and depletes T and/or NK cells expressing KLRG1.

In certain embodiments, the depleting agent is new and not previously known antibody. Known anti-KLRG1 antibodies include clone 13F12F2 (eBioscience), which is a mouse anti-human KLRG1 antibody that binds to the extracellular domain and has demonstrated reactivity against human cells in flow cytometry, clones 14C2A07 (Biolegend) and SA231A2 (Biolegend), which are reported to be anti-human KLRG1 antibodies, clone 13A2 (EBioscience) which is said to bind a similar epitope to clone 13F12F2, clone REA261 (Miltenyi Biotec) which also reportedly binds human KLRG1, and clone 2F1, which is a hamster anti-mouse KLRG1 antibody that some vendors (e.g., Biolegend) report to be reactive against human while others (e.g., Abcam) report reactivity to only mouse. Tests of these antibodies failed to demonstrate reactivity to human KLRG1. (Known anti-E-Cadherin antibodies are described by vendors and include the following examples: clone 67A4, clone MB2, and clone HECD1 (all sold by Abcam); DECMA1 sold by eBioscience; and clone 36/E-cadherin sold by BD Biosciences.) In various embodiments, the KLRG1 antagonist comprises a binding agent that binds to KLRG1 and that is not clone 13F12F2, 14C2A07, SA231A2, or 2F1.

In various embodiments, the KLRG1 depleting agent can be administered by providing an mRNA encoding the depleting agent to the subject. mRNA approaches are being developed by Moderna Therapeutics, CureVac, and the like.

Pharmaceutical Compositions

In various embodiments, the KLRG1 depleting agent (a KLRG1-expressing-cell depleting agent) is prepared as a pharmaceutical composition, for example as a pharmaceutical composition for use as a medicament. In various embodiments, the pharmaceutical composition is for use as a medicament for an autoimmune disease (e.g., rheumatoid arthritis, psoriasis, inclusion body myositis (IBM), multiple sclerosis, ulcerative colitis, lymphocytic colitis, idiopathic thrombocytopenic purpura, or type 1 diabetes); transplant rejection (e.g., kidney rejection, preferably T cell mediated kidney rejection after transplantation); a hematologic malignancy (e.g., a leukemia such as T cell leukemia, NK cell leukemia, large granular lymphocytic leukemia (LGLL), or chronic lymphocytic leukemia (CLL) or a lymphoma such as T cell lymphoma, preferably anaplastic large cell lymphoma); or a solid tumor (e.g., a breast cancer, gastric cancer, ovarian cancer, prostate cancer, glioma, glioblastoma, melanoma, lung cancer, tongue cancer).

One skilled in the art can formulate the KLRG1 depleting agent as a pharmaceutical composition according to known methods.

Pharmaceutical compositions can include a carrier. “Carriers” as used herein can include pharmaceutically acceptable carriers, excipients, or stabilizers that are nontoxic (or relatively non-toxic) to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In various embodiments, the KLRG1 depleting agent is comprised in an injectable formulation, for example, a subcutaneous, intravenous, intramuscular, intrathecal or intraperitoneal injection formulation. Injectable formulations can be aqueous solutions, for example in physiologically compatible buffers such as Hanks' solution, Ringer's solution, or physiological saline buffer. The injectable formulation can contain formulatory agents such as suspending, stabilizing and/or dispersing agents. Alternatively, the KLRG1 depleting agent can be in a dried or powder form for constitution with a suitable vehicle, e.g., sterile pyrogen-free water, before use.

In certain embodiments, the KLRG1 depleting agent is not any of the KLRG1/ligand binding agents disclosed in PCT Application No. PCT/US17/35621.

Treatment and Administration

The invention provides methods comprising administering the KLRG1 depleting agent (a KLRG1-expressing-cell depleting agent) according to any of the aspects or embodiments disclosed herein, or the pharmaceutical composition according to any of the aspects or embodiments disclosed herein, to a subject in need thereof. In various embodiments, the subject is a human. In various embodiments, methods according to the invention are carried out in vivo (e.g., as opposed to ex vivo). As used herein, “treatment” can refer to both therapeutic treatment and prophylactic or preventative measures, wherein the object is to prevent or slow down (lessen) the targeted pathologic condition or disorder. Those in need of treatment can include those already with the disorder, those prone to have the disorder, or those in whom the disorder is to be prevented.

In various embodiments, the invention provides methods for treating a condition associated with overexpression KLRG-1. In a preferred embodiment, the invention targets a subset of cells, for example T cells and/or NK cells that express higher than normal levels of KLRG-1.

In various aspects and embodiments, the invention provides methods for treating an autoimmune disease. The autoimmune disease can be, for example, rheumatoid arthritis, psoriasis, inclusion body myositis (IBM), multiple sclerosis, ulcerative colitis, lymphocytic colitis, idiopathic thrombocytopenic purpura, primary biliary cholangitis, or type 1 diabetes. As described herein, cytotoxic T cells are implicated in the pathogenesis of these diseases. In accordance with the present invention, depletion of such cytotoxic T cells provides a therapeutic benefit.

In various aspects and embodiments, the invention provides methods for treating or preventing transplant rejection. The transplant rejection can be, for example, kidney rejection (e.g., T cell mediated kidney rejection after transplantation).

In various aspects and embodiments, the invention provides methods for treating a hematologic malignancy. The hematologic malignancy can be, for example, a leukemia such as T cell leukemia, NK cell leukemia, large granular lymphocytic leukemia (LGLL), or chronic lymphocytic leukemia (CLL). The hematologic malignancy can be, for example, a lymphoma such as T cell lymphoma, anaplastic large cell lymphoma (ALCL), peripheral T cell lymphoma (PTCL), or angioimmunoblastic T cell lymphoma (AITCL). As described herein, KLRG1 is expressed by T cells in these leukemias and lymphomas to a similar extent by gene expression as normal or activated T cells, the majority of which express KLRG1. In accordance with the present invention, depletion of such cytotoxic T cells provides a therapeutic benefit.

In various aspects and embodiments, the invention provides methods for treating a solid tumor. The solid tumor can be, for example, a breast cancer, gastric cancer, ovarian cancer, prostate cancer, glioma, glioblastoma, melanoma, or lung cancer. KLRG1 is a co-inhibitory receptor present on T and NK cells, and engagement by its ligands results in T and NK cell inhibition. These inhibited T and NK cells furthermore appear to inhibit the function of other T and NK cells (without wishing to be bound by any particular theory, possibly through space occupying or other cell-cell interactions). For example, the binding of KLRG1-expressing T cells to its ligand, E-cadherin, expressed on a cancer cell surface can obstruct non-KLRG1-expressing T cells from reaching the cancer cell surface and killing the cancer cell. Removal of such inhibited KLRG1-expressing-cells (e.g., ineffective T and/or NK cells) can allow properly functioning immune system cells to successfully attack cancer cells. For example, experiments suggest E-cadherin expression on human breast carcinoma cells affects trastuzumab-mediated ADCC through KLRG1 on NK cells (Yamauchi et al., 2011)

“Administration” and “treatment,” as it applies to an animal, human, experimental subject, cell, tissue, organ, or biological fluid, can include contacting an exogenous pharmaceutical, therapeutic agent, diagnostic agent, or composition to the animal, human, subject, cell, tissue, organ, or biological fluid. “Administration” and “treatment” include in vivo, as well as in some embodiments, in vitro or ex vivo treatments. In various embodiments, the methods are carried out in vivo.

“Treat” or “treating” means to administer a therapeutic agent, such as a composition containing any of the depleting agents of the present invention, internally or externally to a subject or patient having one or more disease symptoms, or being suspected of having a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

As such, in various embodiments, the term “effective amount” is a concentration or amount of the KLRG1 depleting agent which results in achieving a particular stated purpose. An “effective amount” of a KLRG1 depleting agent can be determined empirically. Furthermore, a “therapeutically effective amount” is a concentration or amount of a KLRG1 depleting agent which is effective for achieving a stated therapeutic effect. This amount can also be determined empirically.

In various embodiments, the KLRG1 depleting agent can be administered by providing an mRNA encoding the depleting agent to the subject.

In various embodiments, the treatment can prolong the subject's survival. In various embodiments, the treatment can prevent or reduce the progression of the cancer and/or metastasis.

The following examples are illustrative and not restrictive. Many variations of the technology will become apparent to those of skill in the art upon review of this disclosure. The scope of the technology should, therefore, be determined not with reference to the examples, but instead should be determined with reference to the appended claims along with their full scope of equivalents.

### EXAMPLES

### Example 1: Antibodies to KLRG1

Antibodies binding KLRG1 were generated by immunization of mice with purified recombinant protein antigens: Human-KLRG1 ECD Isotype 1 (SEQ ID NO: 1), Human-KLRG1 ECD Isotype 2 (SEQ ID NO:2) and Cytomolgus-KLRG1 ECD (SEQ ID NO:3). Balb/c and SJL mice were immunized every 2 weeks with recombinant KLRG1 protein and sera collected for testing after the second and fourth immunization.

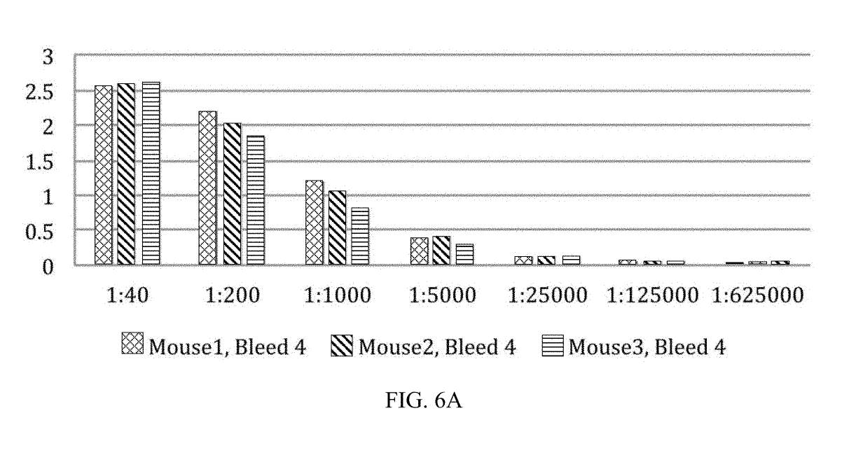

FIG. 6A show a specific serum response to the cynomolgus KLRG1 in the immunized mice. FIG. 6B show a specific serum response to the human KLRG1 in the immunized mice. Anti-KLRG1 antibody activity was measured by ELISA. The response was shown to be mediated by production of antibodies in the mouse recognizing human and cynomolgus KLRG1.

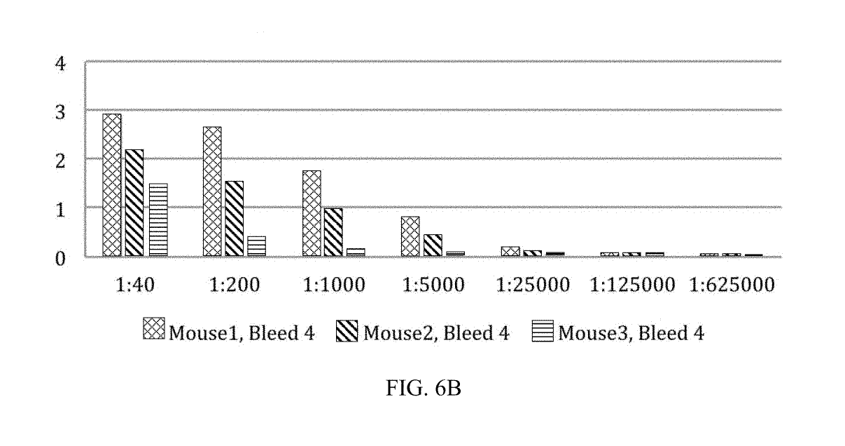

FIG. 7 shows a dose-dependent binding curve of 9 hybridoma clones isolated from immunized mice. ELISA was performed by first immobilizing human KLRG1 (SEQ ID NO:2) on immunosorbent 96-well plates, followed by exposure to a dose-dependent titration of antibodies. Bound antibodies were visualized by anti-mouse-HRP conjugated detection. Thus, FIG. 7 shows that splenocytes isolated from the spleens of immunized mice are able to produce antibodies that recognize KLRG1.

### Example 2: Gene Expression of KLRG1 is Higher Among CD8+ and NK Cells than CD4+ T Cells, and KLRG1 Expression Correlates with CD8+ T Cell Cytotoxic Potential

Cytotoxic cells consist of CD8+ T cells and NK cells. Analysis via abstraction from published figures of flow cytometry data from healthy donors in publications with PubMed ID#s (PMID) of Ser. No. 12/393,723, 20394788, 23966413, 26583066, and 27566818 demonstrates the greater percentage of KLRG1+ cells amongst CD8+(relative-fold 2.5) T cells and CD56+NK cells (relative-fold 2.4) compared to CD4+T helper cells (relative-fold 1.0) (FIG. 1). In response to antigen exposure, CD8+ cytotoxic T cells differentiate from naïve cells to increasingly potent effector cells, characterized by central memory (TCM), effector memory (TEM) and effector (TEMRA) cells. The cytotoxic potential of CD8+ T cells within these differentiation subsets, as reflected by gene expression of cytotoxic molecules granzymes and cytokines, is highly correlated with proportions of KLRG1+ cells within these subsets by flow cytometry (FIGS. 2A and 2B). Gene expression analysis in FIG. 2A was performed on microarray data available in dataset E-TABM-40 of the Array Express database of the European Bioinformatics Institute. Flow cytometry data in FIG. 2B was abstracted from published figures and tables present in a range of publications as indicated, specifically those with PubMed ID#s (PMID) of Ser. No. 12/393,723, 16140789, 18657274, 22347406, 24022692, 24391639, and 26611787, and an online published thesis at discovery.ucl.ac.uk/1317772/1/1317772.pdf.

### Example 3: Inclusion Body Myositis (IBM)

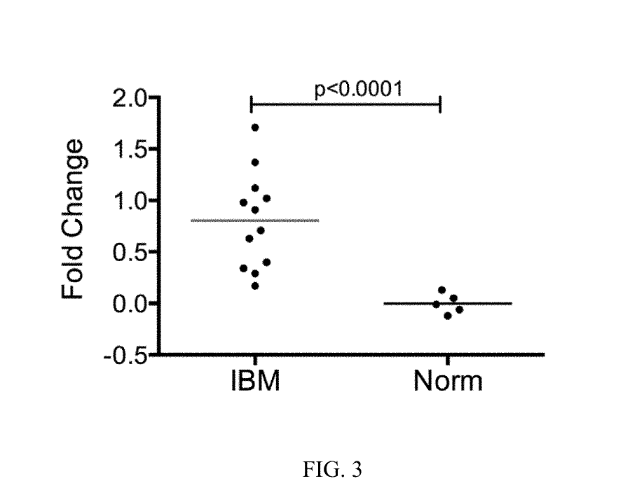

FIG. 3 shows analysis of microarray data from the Gene Expression Omnibus (GEO) dataset GSE39454. Increased expression of KLRG1 is observed in IBM muscle compared to normal muscle.

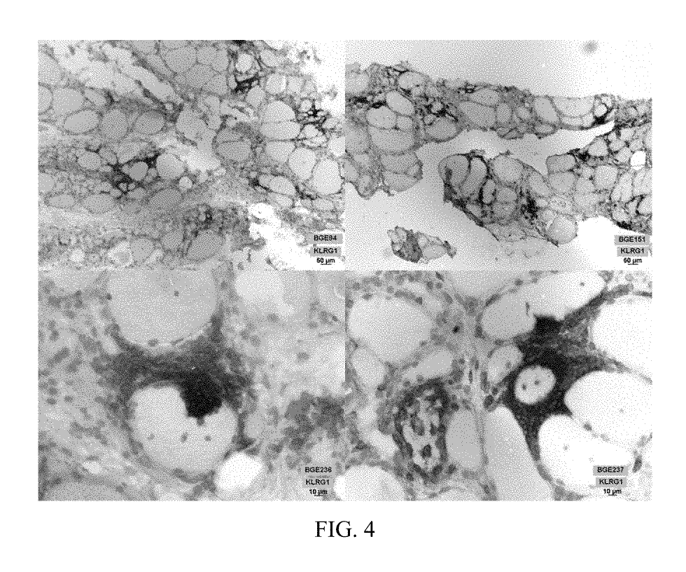

FIG. 4 shows immunohistochemical staining of muscle biopsy sample from 20 patients with IBM. Representative immunohistochemical staining images from four patients are shown in FIG. 4, which shows KLRG1+ infiltrating cells (stained black) attacking muscle fibers in all patients. Isotype controls were used as negative controls (not shown).

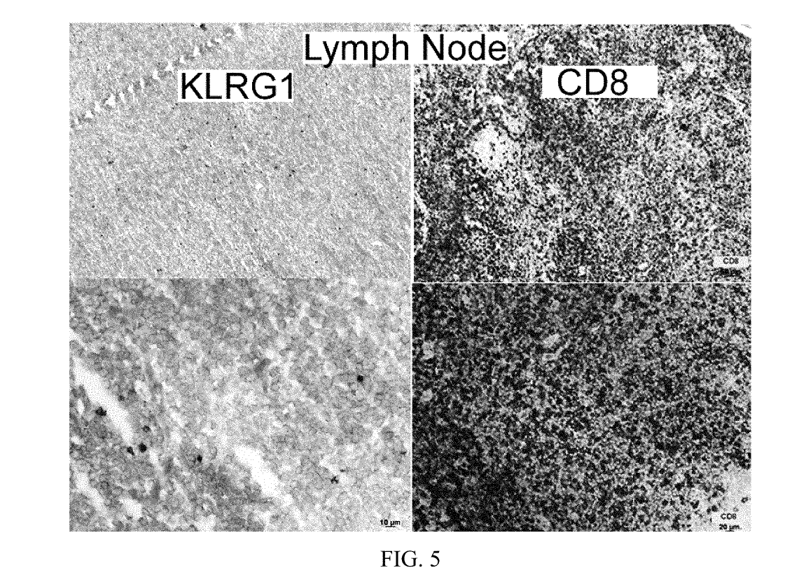

FIG. 5 shows limited expression of KLRG1 on CD8+ T cells (stained black) in lymph node, indicating predicted relative sparing of protective memory T cells after KLRG1-expressing cell depletion. Representative staining images are shown of two human lymph node samples. Isotype controls were used as negative controls (not shown).

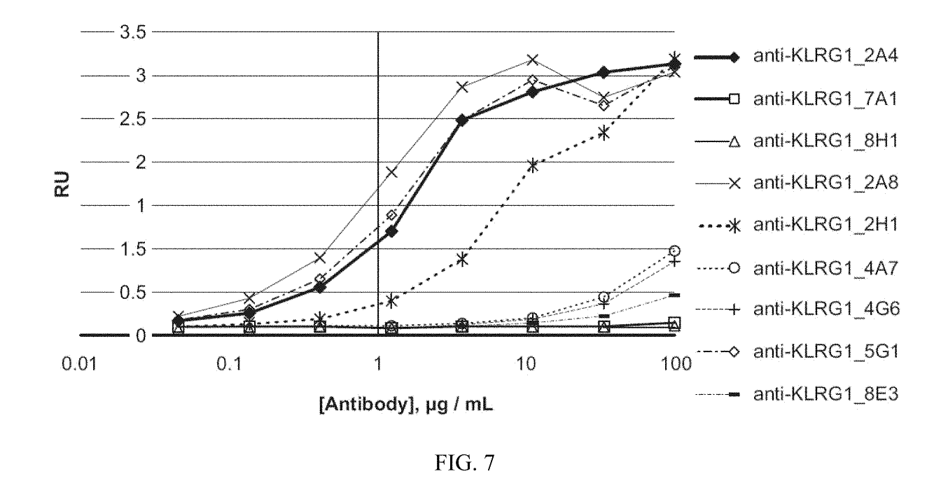

FIGS. 8A and 8B show representative example flow cytometry results of IBM patient (FIG. 8A) and healthy donor (FIG. 8B). Increased numbers of CD8+ KLRG1+ T cells were observed in IBM patients (FIG. 8A) compared to healthy donors (FIG. 8B). Mean age for each group was 65. FIG. 8C shows average % total blood lymphocytes of IBM patients and healthy donors.

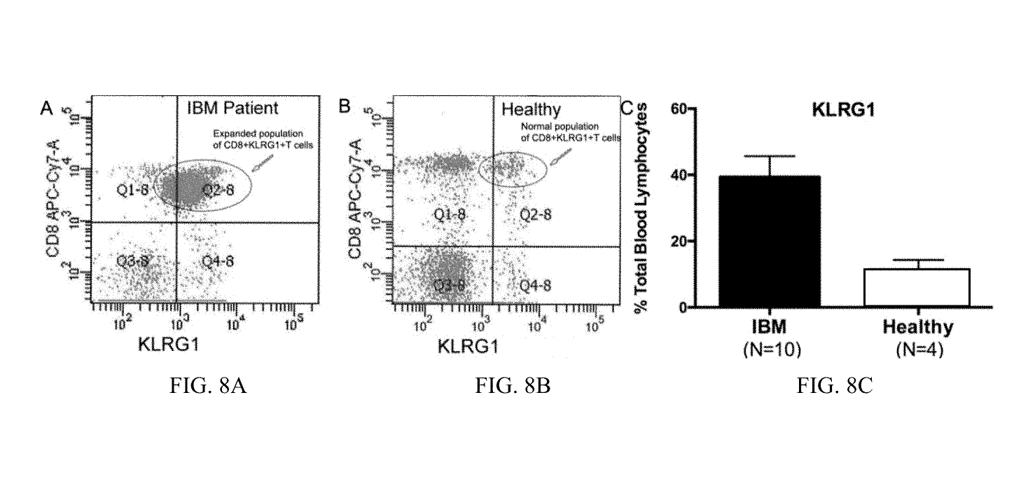

FIGS. 9A and 9B show, by flow cytometry, a proportion of CD8+CD57+ leukemic cells (shown as P2 in FIG. 9A) express KLRG1 (FIG. 9B, cells gated from FIG. 9A as indicated) in a patient with IBM and T cell large granular lymphocytic leukemia (T-LGLL).

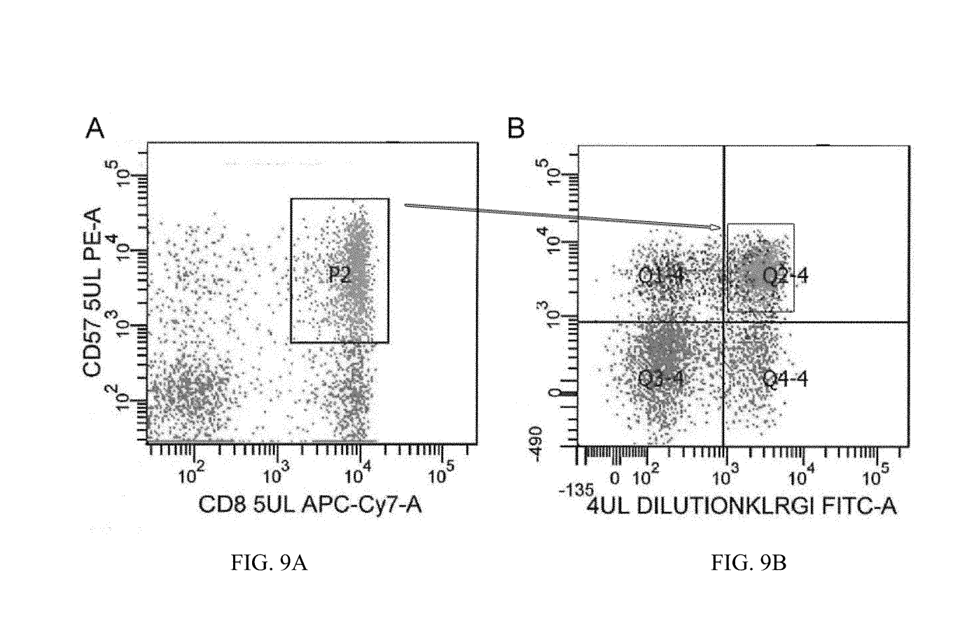

FIGS. 10A and O1B show KLRG1+ infiltrating cells (stained black) attacking muscle by immunohistochemistry in a patient with IBM and T-LGLL and in a patient with IBM and chronic lymphocytic leukemia, respectively. Isotype controls were used as negative controls (not shown).

### Example 4: Ulcerative Colitis

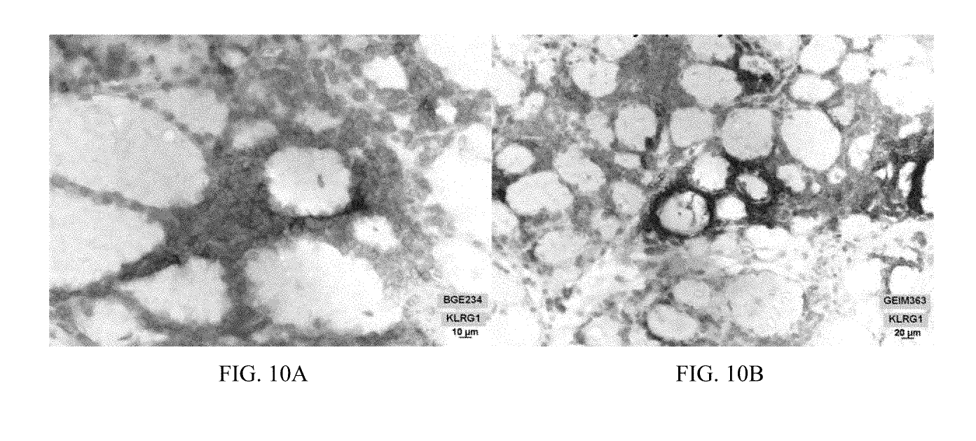

FIG. 11 shows analysis of expression data (E-GEOD-59071) from intestinal biopsies from 74 patients with active ulcerative colitis compared to 5 patients with normal intestine. Increased expression of KLRG1 is observed in intestinal biopsies from patients with active ulcerative colitis compared to patients with normal intestine (1.27 fold ratio). Dataset obtained from ArrayExpress database at the European Bioinformatics Institute and analyzed for KLRG1 expression. Accordingly, ulcerative colitis is an attractive target for therapies according to the present invention.

### Example 5: Production of Antibodies that Bind Human KLRG1

Antibodies that bind to the extracellular portion of KLRG1 can be produced by several techniques including but not limited to: mouse hybridoma technology, phage display, yeast display, retrocyte display, humanized mouse technology, ribosome display. Other and additional methods are known in the art and may be developed in connection with the application of the present invention.

For example, mouse hybridoma technology can be used to generate antibodies that bind to KLRG1 and deplete KLRG1-expressing cells. Strains of mice commonly used for antibody generation can be used, for example, Balb/c or SJL strains. Multiple mice can be injected repeatedly at 2 weeks intervals with antigen to produce an immune response. Several forms of the antigen can be injected either alone or in combination and with the addition of adjuvants such as KLH (keyhole limpet hemocyanin) known to enhance the immune response of the host to foreign antigens. Antigens can be in the form of purified recombinant KLRG1, cDNA coding for KLRG1, cells expressing KLRG1 on their surface or peptides derived from the sequence of KLRG1.

After every administration of antigens to the mice, the immune response against KLRG1 can be monitored by ELISA titer. The ELISA can be carried out by first immobilizing recombinant KLRG1 on suitable ELISA microtiter plates. After 12 hour incubation, the plates can be washed with phosphate saline and blocked with 1% solution of BSA in phosphate buffered saline. Sera derived from immunized mice can be serially diluted in phosphate buffered saline and allowed to interact with the surface bound antigen in the microtiter plates. Excess sera can be washed away and the amount of binding can be visualized using standard techniques such as addition of anti-mouse antibody conjugated to HRP. Mice can be boosted with antigen until a sufficiently high level of signal can be detected in their serum at which point the spleen can be removed from the mice. Splenocytes derived from immunized mice can be fused with myeloma derived (SP2/0) using standard protocols in use in the field. The resulting hybridoma cells can express and secrete antibodies that can be tested for binding to recombinant KLRG1 using ELISA and to cell expressed KLRG1 using FACS. Hybridoma cell lines that produce antibodies with desired binding characteristics can be sub-cloned and the variable regions of the antibody sequenced. Recombinant antibodies using these variable mouse regions and human constant regions can produced by standard techniques, and can be evaluated in functional assays. (e.g., for binding and/or depleting activity).

### Example 6: Depletion of CD8+CD57+ Terminally Differentiated Cells Using Anti-KLRG1 Antibodies

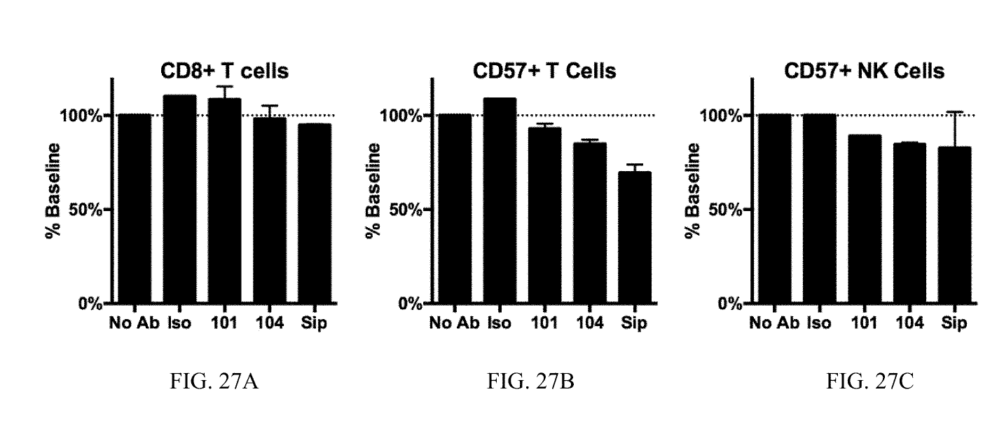

FIGS. 27A-C show flow cytometry of human whole blood collected in heparin comparing baseline to 2.5 hours of incubation with IgG1 isotype antibody (Iso), anti-KLRG1 mouse/human IgG1 chimeric antibody CHI101(101), anti-KLRG1 mouse/human IgG1 chimeric antibody CHI104 (104), and anti-CD2 antibody siplizumab (Sip), a known potent T cell depleting antibody used as a positive control. Antibody CHI101 and CHI104 resulted in selective depletion of CD8+CD57+ T cells and CD8+CD57+NK cells.

### Example 7: Tongue Carcinoma

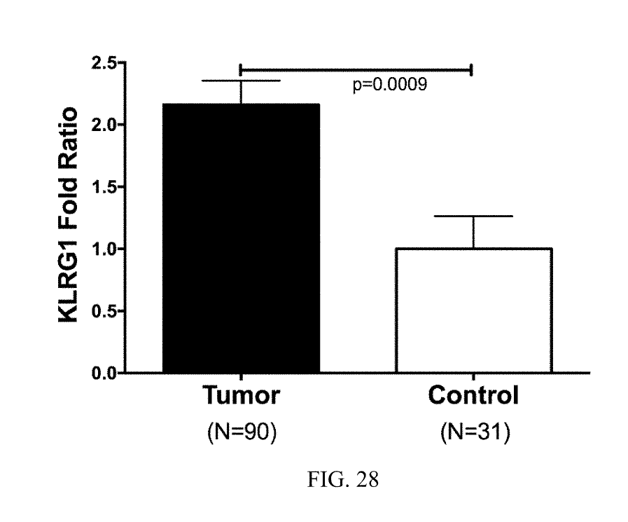

FIG. 28 shows analysis of expression data (GSE34115) from tongue biopsies from 90 patients with tongue carcinoma compared to 31 patients without tongue carcinoma. Increased expression of KLRG1 (2.16-fold ratio) was observed in tongue biopsies from patients with tongue carcinoma. Dataset was obtained from ArrayExpress database at the European Bioinformatics Institute and analyzed for KLRG1 expression. Accordingly, tongue carcinoma is an attractive target for therapies according to the present invention.

### Example 8: Idiopathic Thrombocytopenic Purpura

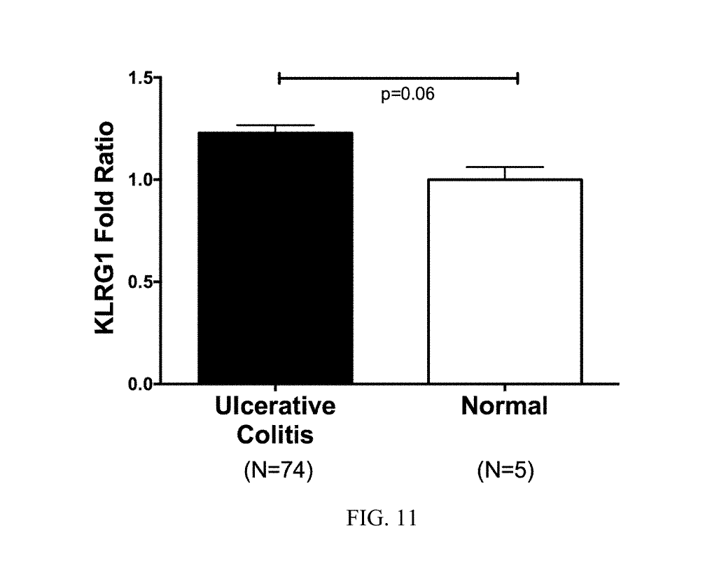

FIG. 12 shows analysis of blood CD3+ T cell expression data (GSE574) from 2 patients with ITP compared to 2 healthy persons. Increased expression of KLRG1 (2.69-fold ratio) was observed in CD3+ T cells of patients with ITP. Dataset was obtained from Gene Expression Omnibus database at the National Center for Bioinformatics and analyzed for KLRG1 expression. Accordingly, idiopathic thrombocytopenic purpura is a particularly attractive target for therapies according to the present invention.

### Example 9: Lymphocytic Colitis

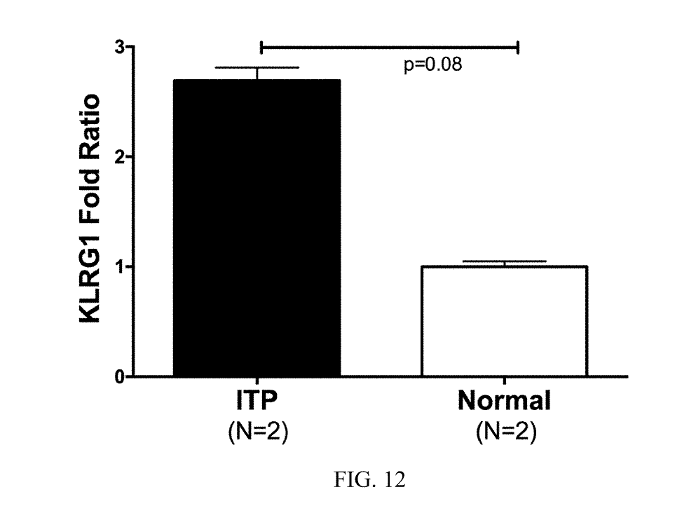

FIG. 13 shows analysis of expression data (GSE65107) from colon biopsies of 4 patients with lymphocytic colitis compared to colon biopsies of 4 healthy persons. Increased expression of KLRG1 (3.8-fold ratio) was observed in colon biopsies of patients with lymphocytic colitis. Dataset was obtained from Gene Expression Omnibus database at the National Center for Bioinformatics and analyzed for KLRG1 expression. Accordingly, lymphocytic colitis is a particularly attractive target for therapies according to the present invention.

### Example 10: Renal Transplant Rejection

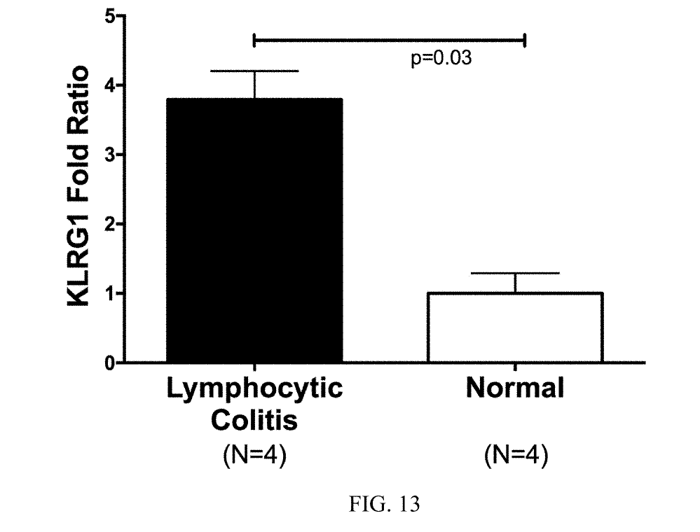

FIG. 14 shows analysis of expression data (GSE36059) from patients with renal transplant rejection compared to nephrectomies. Increased expression of KLRG1 (2.69-fold ratio) was observed in renal transplantation rejection kidney biopsies. Dataset obtained from Gene Expression Omnibus database at the National Center for Bioinformatics and analyzed for KLRG1 expression, with additional analysis through NextBio. Accordingly, renal transplant rejection is a particularly attractive target for therapies according to the present invention.

### Example 11: Anaplastic Large Cell Lymphoma

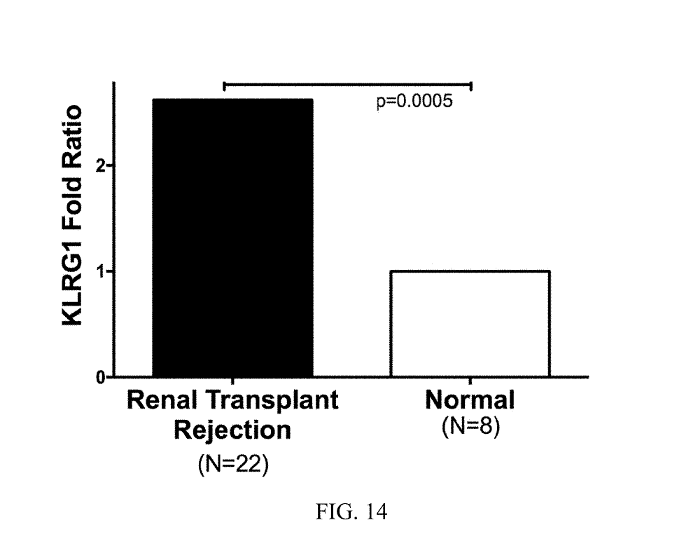

FIG. 15 shows analysis of expression data (GSE6338) from lymph node biopsies from 6 patients with anaplastic large cell lymphoma compared to normal CD4+ T cells from lymph node in 5 patients. Increased expression of KLRG1 (3.55-fold ratio) was observed in lymphnode biopsies from patients with anaplastic large cell lymphoma. Dataset obtained from Gene Expression Omnibus database at the National Center for Bioinformatics and analyzed for KLRG1 expression. Accordingly, anaplastic large cell lymphoma is a particularly attractive target for therapies according to the present invention.

### Example 12: Rheumatoid Arthritis

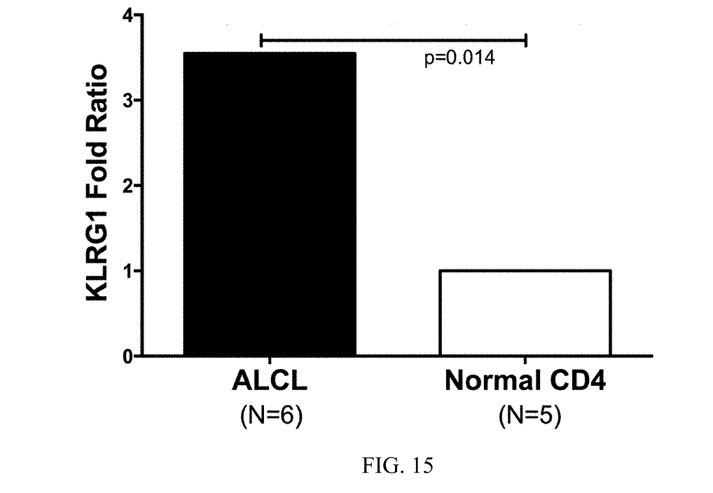

FIG. 16 shows analysis of expression data (GSE1919) from synovium biopsies from patients with rheumatoid arthritis compared to normal subjects. Increased expression of KLRG1 (3.55-fold ratio) was observed in synovium biopsies from patients with rheumatoid arthritis. Dataset obtained from Gene Expression Omnibus database at the National Center for Bioinformatics and analyzed for KLRG1 expression. Accordingly, rheumatoid arthritis is a particularly attractive target for therapies according to the present invention.

### Example 13: Psoriasis

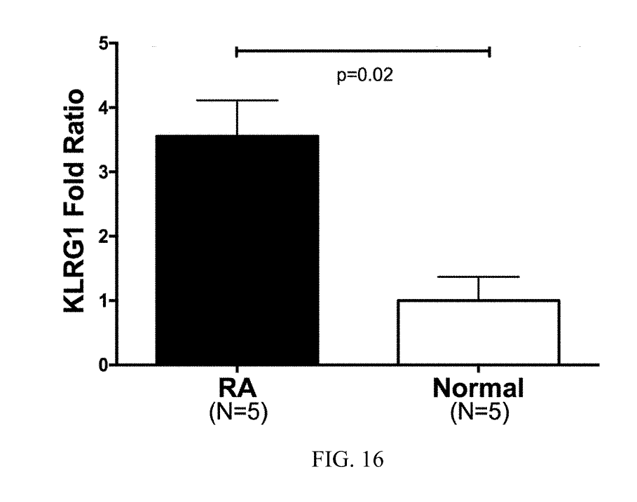

FIG. 17 shows analysis of expression data (GSE52471) from skin biopsies from patients with psoriasis compared to normal subjects. Increased expression of KLRG1 (1.14-fold ratio) was observed in skin biopsies from patients with psoriasis. Dataset obtained from Gene Expression Omnibus database at the National Center for Bioinformatics and analyzed for KLRG1 expression. Accordingly, psoriasis is a particularly attractive target for therapies according to the present invention.

### Example 14: Primary Biliary Cholangitis

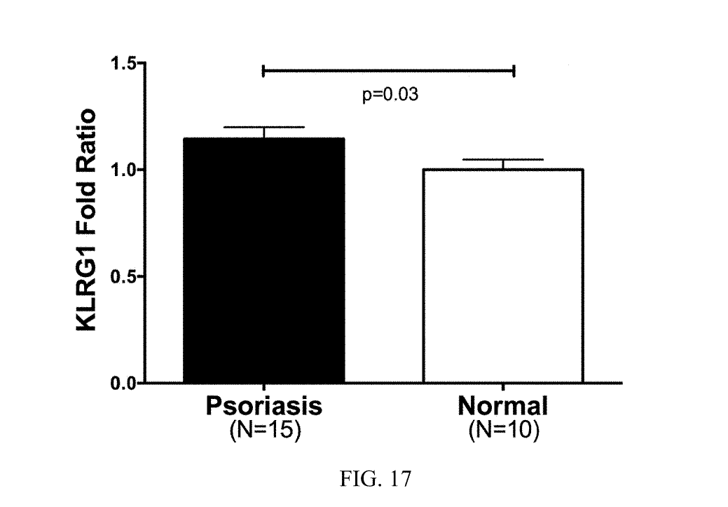

FIG. 18 shows analysis of expression data (GSE79850) from liver biopsies from patients with primary biliary cholangitis eventually requiring liver transplantation compared to normal subjects. Increased expression of KLRG1 (6.03-fold ratio) was observed in liver biopsies from patients with primary biliary cholangitis. Dataset obtained from Gene Expression Omnibus database at the National Center for Bioinformatics and analyzed for KLRG1 expression. Accordingly, primary biliary cholangitis is a particularly attractive target for therapies according to the present invention.

### Example 15: Type 1 Diabetes

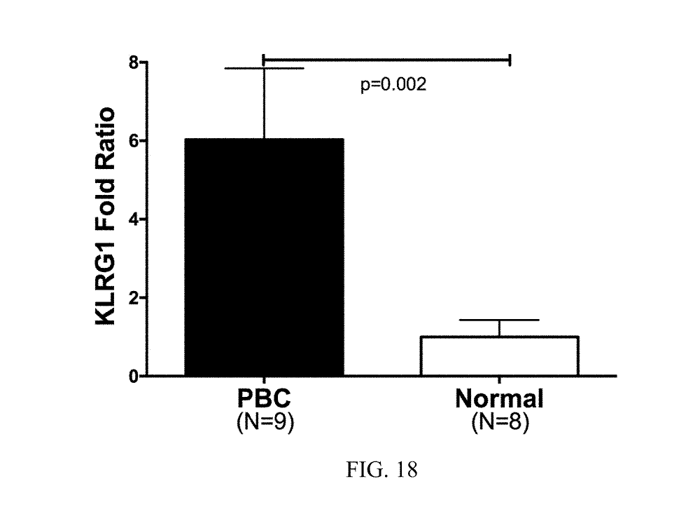

FIG. 19 shows analysis of expression data (GSE72492) from pancreas from patients with type 1 diabetes compared to normal subjects. Increased expression of KLRG1 (1.66-fold ratio) was observed in pancreas from patients with type 1 diabetes. Dataset obtained from Gene Expression Omnibus database at the National Center for Bioinformatics and analyzed for KLRG1 expression. Accordingly, type 1 diabetes is a particularly attractive target for therapies according to the present invention.

### Example 16: Large Granular Lymphocytic Leukemia

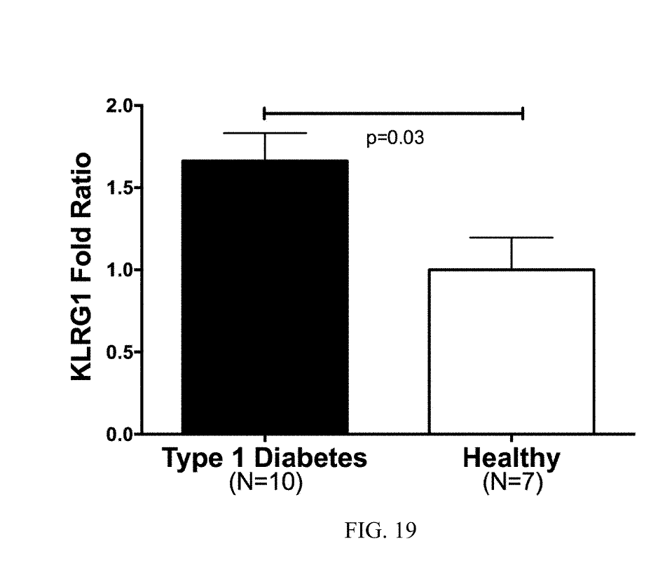

FIG. 20 shows analysis of expression data (GSE10631) from blood from patients with T cell large granular lymphocytic leukemia compared to normal subjects. Similar or increased expression of KLRG1 (1.39-fold ratio) was observed in blood from patients with T cell large granular lymphocytic leukemia. Dataset obtained from Gene Expression Omnibus database at the National Center for Bioinformatics and analyzed for KLRG1 expression. Accordingly, these T cell large granular lymphocytic leukemia samples contain KLRG1 and T cell large granular lympchocytic leukemia is a particularly attractive target for therapies according to the present invention.

### Example 17: T Cell Leukemias and Lymphomas

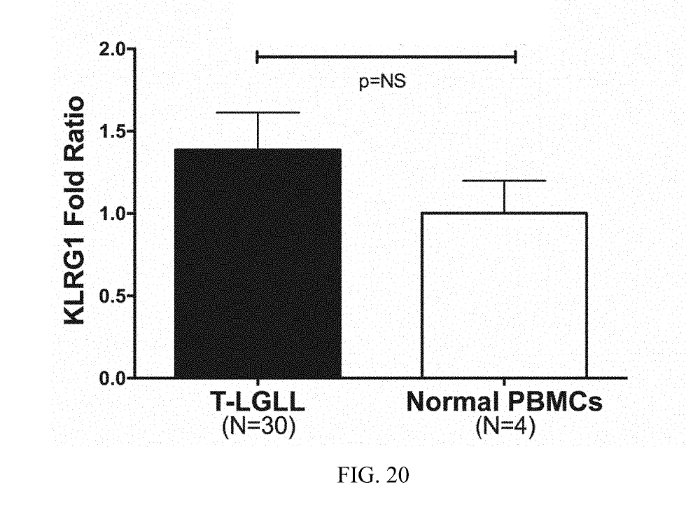

FIG. 21 shows analysis of expression data (GSE19069) from lymphoma biopsies from patients with a variety of T cell leukemias and lymphomas, including anaplastic large cell lymphoma (ALCL), angioimmunoblastic T-cell lymphoma (AITCL), and peripheral T-cell lymphoma (PTCL). Similar expression of KLRG1 was observed in the various T cell lymphomas compared to normal T cells (range 0.45- to 1.52-fold increase). Dataset obtained from Gene Expression Omnibus database at the National Center for Bioinformatics and analyzed for KLRG1 expression. Accordingly, these T cell lymphomas contain KLRG1-expressing T cells and are particularly attractive targets for therapies according to the present invention.

### Example 18: Multiple Sclerosis

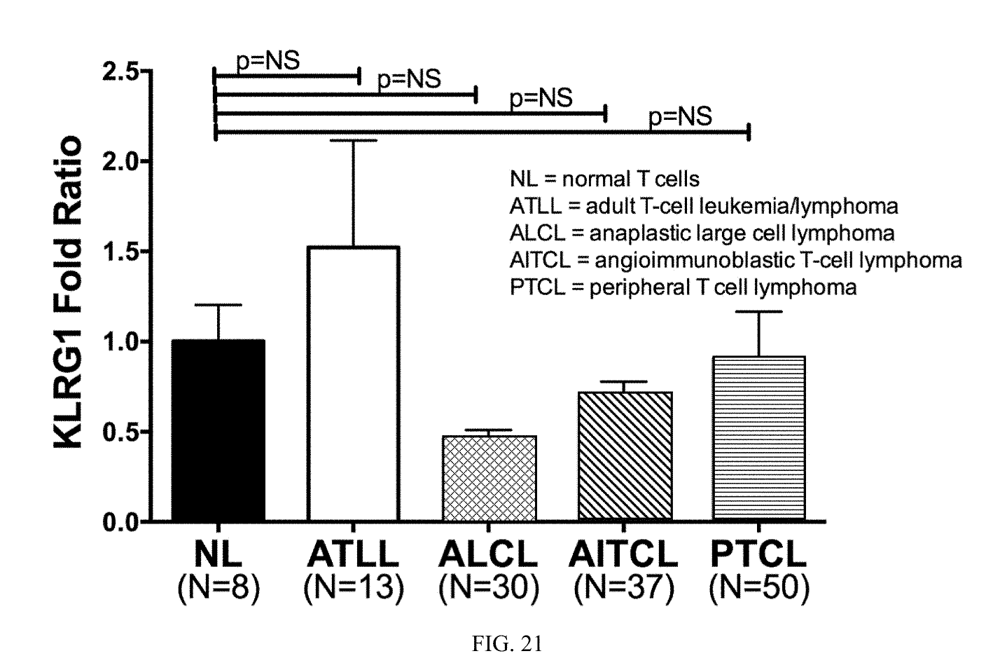

FIG. 22 shows analysis of expression data (GSE5839) from brain biopsies from patients with a multiple sclerosis and compared to normal brain. Elevated expression of KLRG1 is observed compared to control brain (1.23-fold). Dataset obtained from Gene Expression Omnibus database at the National Center for Bioinformatics and analyzed for KLRG1 expression. Accordingly, multiple sclerosis is a particularly attractive target for therapies according to the present invention.

### Example 19: Increased Expression of KLRG1 in Human Cancer

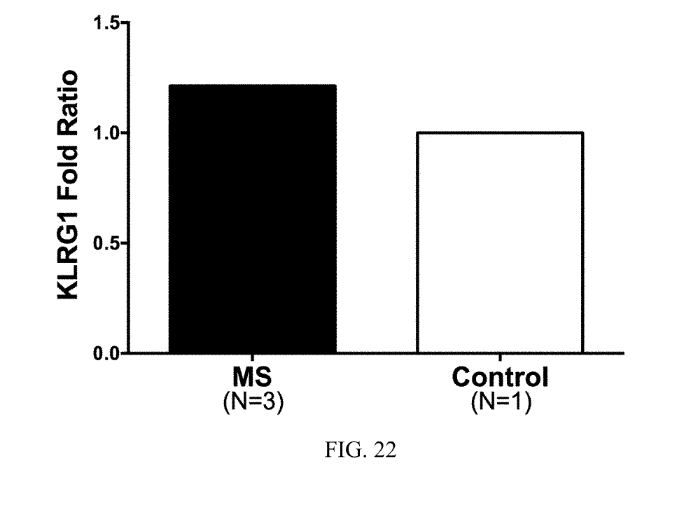

FIG. 23 shows KLRG1 is expressed by tumor infiltrating lymphocytes in a wide variety of cancer, as detected by RNAseq expression. TCGA raw RNAseq data was downloaded from the TCGA database. Cancer tissue samples (N=9,755) across 32 cancer types were analyzed. X-axis denotes log 2 RPKM values, Y-axis contains cancer types, each dot represents the level of KLRG1 expression in a single cancer tissue sample.

FIG. 23 shows expression of KLRG1 in tumor samples in many cancer types. Cancer types listed from left to right are: uveal melanoma, uterine carcinoma, uterine carcinosarcoma, thyroid carcinoma, thymoma, testicular germ cell tumor, melanoma, sarcoma, rectal adenocarcinoma, prostate cancer, pheochromocytoma, pancreatic adenocarcinoma, ovarian cysadenocarcinoma, mesothelioma, lung squamous cell carcinoma, lung adenocarcinoma, liver hepatocellular carcinoma, kidney papillary cell carcinoma, kidney clear cell carcinoma, kidney chromophobe, head and neck squamous cell carcinoma, glioblastoma multiforme, diffuse large B-cell lymphoma, colon adenocarcinoma, cholangiocarcinoma, cervical and endocervical cancer, breast invasive carcinoma, brain low grade glioma, bladder cancer, adrenocortical cancer, and acute myeloid leukemia. Accordingly, such cancers are particularly attractive targets for therapies according to the present invention.

### Example 20: Melanoma

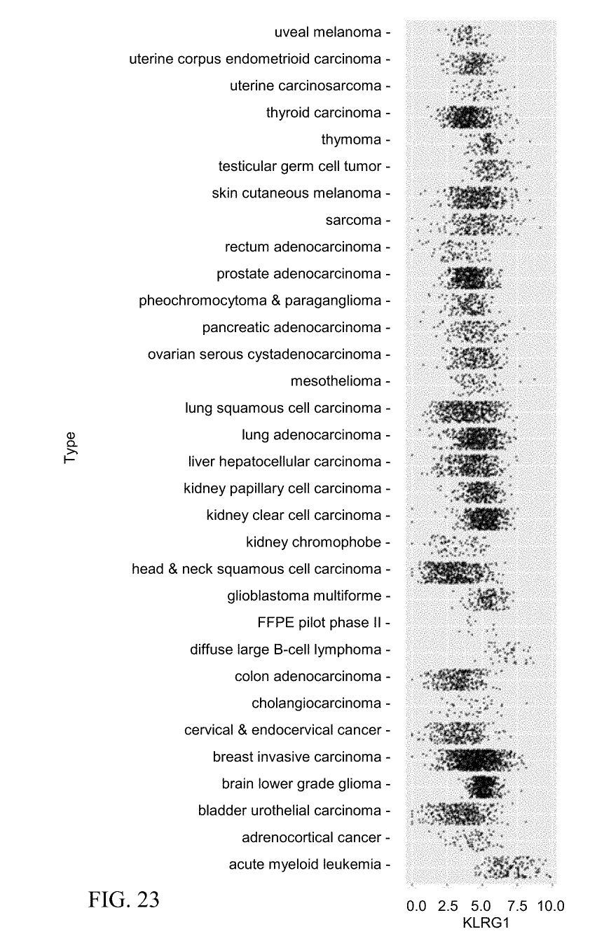

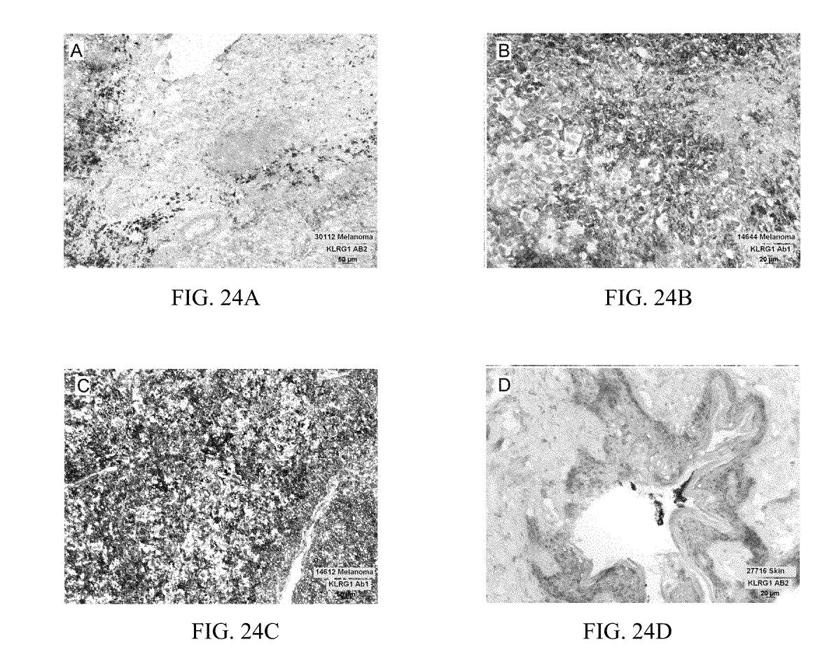

FIGS. 24A-C show immunohistochemistry of human melanoma biopsies from 3 patients. The results demonstrate abundant KLRG1+ cells (stained black) infiltrating tumor. Isotype controls were used as negative controls (not shown). FIG. 24D shows absence of KLRG1+ cells in normal skin. The presence of KLRG1+ cells in tumor tissue render melanoma a particularly attractive target for therapies according to the present invention.

### Example 21: Renal Cell Carcinoma

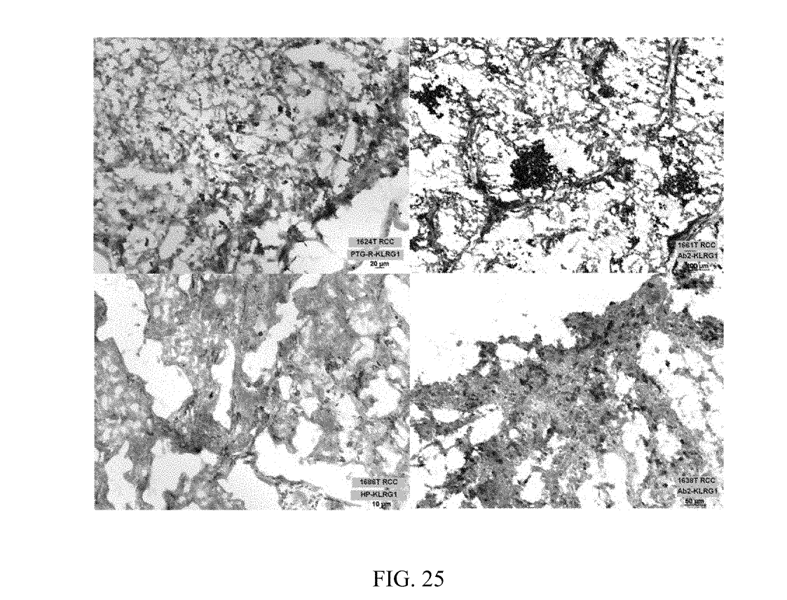

FIG. 25 shows immunohistochemistry of human renal cell carcinoma biopsies from 4 patients. The results demonstrate abundant KLRG1+ cells (stained black) infiltrating tumor. Isotype controls were used as negative controls (not shown). The presence of KLRG1+ cells in tumor tissue render renal cell carcinoma a particularly attractive target for therapies according to the present invention.

### Example 22: Non-Small Cell Lung Cancer

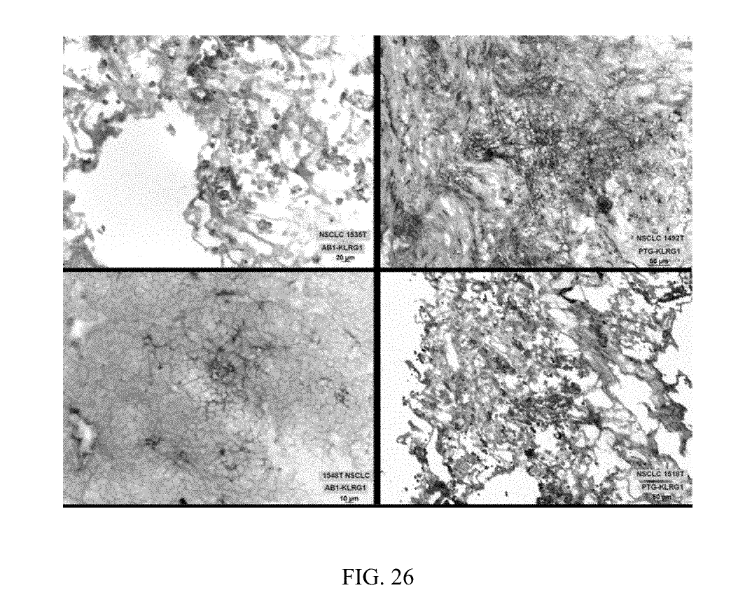

FIG. 26 shows immunohistochemistry of human non-small cell lung cancer biopsies from 4 patients. Abundant KLRG1+ cells (stained black) infiltrating tumor were observed. Isotype controls were used as negative controls (not shown). The presence of KLRG1+ cells in tumor tissue render non-small cell lung cancer a particularly attractive target for therapies according to the present invention.

### REFERENCES

Akbar A N, Henson S M. Are senescence and exhaustion intertwined or unrelated processes that compromise immunity? Nat Rev Immunol. 2011; 11(4):289-95.
Amemiya K, Granger R P, Dalakas M C. Clonal restriction of T-cell receptor expression by infiltrating lymphocytes in inclusion body myositis persists over time. Studies in repeated muscle biopsies. Brain: a journal of neurology. 2000; 123 (Pt 10):2030-9.
Apetoh L, Smyth M J, Drake C G, Abastado J P, Apte R N, Ayyoub M, et al. Consensus nomenclature for CD8 T cell phenotypes in cancer. Oncoimmunology. 2015; 4(4):e998538.
Arahata K, Engel A G. Monoclonal antibody analysis of mononuclear cells in myopathies. I: Quantitation of subsets according to diagnosis and sites of accumulation and demonstration and counts of muscle fibers invaded by T cells. Ann Neurol. 1984; 16(2):193-208.
Arahata K, Engel A G. Monoclonal antibody analysis of mononuclear cells in myopathies. III: Immunoelectron microscopy aspects of cell-mediated muscle fiber injury. Ann Neurol. 1986; 19(2):112-25.
Arahata K, Engel A G. Monoclonal antibody analysis of mononuclear cells in myopathies. IV: Cell-mediated cytotoxicity and muscle fiber necrosis. Ann Neurol. 1988; 23(2):168-73.
Attig S, Hennenlotter J, Pawelec G, Klein G, Koch S D, Pircher H, et al. Simultaneous infiltration of polyfunctional effector and suppressor T cells into renal cell carcinomas. Cancer Res. 2009; 69(21):8412-9.
Betjes M G, Meijers R W, de Wit E A, Weimar W, Litjens N H. Terminally differentiated CD8+ Temra cells are associated with the risk for acute kidney allograft rejection. Transplantation. 2012; 94(1):63-9.
Bisping G, Lugering N, Lutke-Brintrup S, Pauels H G, Schurmann G, Domschke W, et al. Patients with inflammatory bowel disease (IBD) reveal increased induction capacity of intracellular interferon-gamma (IFN-gamma) in peripheral CD8+ lymphocytes co-cultured with intestinal epithelial cells. Clinical and experimental immunology. 2001; 123(1):15-22.
Blanco P, Viallard J F, Pellegrin J L, Moreau J F. Cytotoxic T lymphocytes and autoimmunity. Current opinion in rheumatology. 2005; 17(6):731-4.
Brunner S M, Rubner C, Kesselring R, Martin M, Griesshammer E, Ruemmele P, et al. Tumor-infiltrating, interleukin-33-producing effector-memory CD8(+) T cells in resected hepatocellular carcinoma prolong patient survival. Hepatology. 2015; 61(6):1957-67.
Bueno V, Pestana J O. The role of CD8+ T cells during allograft rejection. Brazilian journal of medical and biological research=Revista brasileira de pesquisas medicas e biologicas/Sociedade Brasileira de Biofisica [et al]. 2002; 35(11):1247-58.
Carvalheiro H, Duarte C, Silva-Cardoso S, daSilva JA, Souto-Carneiro M M. CD8 T cell profiles in patients with rheumatoid arthritis and their relationship to disease activity. Arthritis & rheumatology. 2014; 37(2):363-71.
D'Asaro M, Dieli F, Caccamo N, Musso M, Porretto F, Salerno A. Increase of CCR7-CD45RA+CD8 T cells (T(EMRA)) in chronic graft-versus-host disease. Leukemia. 2006; 20(3):545-7.
Faustman D L, Davis M. The primacy of CD8 T lymphocytes in type 1 diabetes and implications for therapies. Journal of molecular medicine. 2009; 87(12):1173-8.
Friese M A, Fugger L. Pathogenic CD8(+) T cells in multiple sclerosis. Ann Neurol. 2009; 66(2):132-41.
Greenberg S A, Pinkus J L, Amato A A, Kristensen T, Dorfman D M. Association of inclusion body myositis with T cell large granular lymphocytic leukaemia. Brain: a journal of neurology. 2016; 139:1348-1360.
Guthmann M D, Tal M, Pecht I. A new member of the C-type lectin family is a modulator of the mast cell secretory response. Int Arch Allergy Immunol. 1995; 107(1-3):82-6.
Hijnen D, Knol E F, Gent Y Y, Giovannone B, Beijn S J, Kupper T S, et al. CD8(+) T cells in the lesional skin of atopic dermatitis and psoriasis patients are an important source of IFN-gamma, IL-13, IL-17, and IL-22. J Invest Dermatol. 2013; 133(4):973-9.
Hofmann M, Schweier O, Pircher H. Different inhibitory capacities of human and mouse KLRG1 are linked to distinct disulfide-mediated oligomerizations. Eur J Immunol. 2012; 42(9):2484-90.
Kita H. Autoreactive CD8-specific T-cell response in primary biliary cirrhosis. Hepatology research: the official journal of the Japan Society of Hepatology. 2007; 37 Suppl 3:S402-5.
Lamy T, Loughran T P, Jr. How I treat LGL leukemia. Blood. 2011; 117(10):2764-74.
Legat A, Speiser D E, Pircher H, Zehn D, Fuertes Marraco S A. Inhibitory Receptor Expression Depends More Dominantly on Differentiation and Activation than “Exhaustion” of Human CD8 T Cells. Front Immunol. 2013; 4:455.
Melis L, Van Praet L, Pircher H, Venken K, Elewaut D. Senescence marker killer cell lectin-like receptor G1 (KLRG1) contributes to TNF-alpha production by interaction with its soluble E-cadherin ligand in chronically inflamed joints. Ann Rheum Dis. 2014; 73(6):1223-31.
Muller S, Lory J, Corazza N, Griffiths G M, Z'Graggen K, Mazzucchelli L, et al. Activated CD4+ and CD8+ cytotoxic cells are present in increased numbers in the intestinal mucosa from patients with active inflammatory bowel disease. The American journal of pathology. 1998; 152(1):261-8.
Okajima M, Wada T, Nishida M, Yokoyama T, Nakayama Y, Hashida Y, et al. Analysis of T cell receptor Vbeta diversity in peripheral CD4 and CD8 T lymphocytes in patients with autoimmune thyroid diseases. Clinical and experimental immunology. 2009; 155(2):166-72.
Schirmer M, Goldberger C, Wurzner R, Duftner C, Pfeiffer K P, Clausen J, et al. Circulating cytotoxic CD8(+) CD28(−) T cells in ankylosing spondylitis. Arthritis research. 2002; 4(1):71-6.
Takata K, Hong M E, Sitthinamsuwan P, Loong F, Tan S Y, Liau J Y, et al. Primary cutaneous NK/T-cell lymphoma, nasal type and CD56-positive peripheral T-cell lymphoma: a cellular lineage and clinicopathologic study of 60 patients from Asia. The American journal of surgical pathology. 2015; 39(1):1-12.
Tessmer M S, Fugere C, Stevenaert F, Naidenko O V, Chong H J, Leclercq G, et al. KLRG1 binds cadherins and preferentially associates with SHIP-1. International immunology. 2007; 19(4):391-400.
Trevino M A, Teixeiro E, Bragado R. CD8+ T cells oligoclonally expanded in synovial fluid at onset of spondyloarthropathy selectively proliferate in response to self-antigens: characterization of cell specificities in nonclonal populations. The Journal of rheumatology. 2004; 31(10):1962-72.
Voehringer D, Koschella M, Pircher H. Lack of proliferative capacity of human effector and memory T cells expressing killer cell lectinlike receptor G1 (KLRG1). Blood. 2002; 100(10):3698-702.
Xing L, Dai Z, Jabbari A, Cerise J E, Higgins C A, Gong W, et al. Alopecia areata is driven by cytotoxic T lymphocytes and is reversed by JAK inhibition. Nat Med. 2014; 20(9):1043-9.
Yamauchi C, Fujii S, Kimura T, Kuwata T, Wada N, Mukai H, Matsumoto N, Fukayama M, Ochiai A. E-cadherin expression on human carcinoma cell affects trastuzumab-mediated antibody-dependent cellular cytotoxicity through killer cell lectin-like receptor G1 on natural killer cells. Int J Cancer. 2011; 128(9):2125-37.
Yu H G, Lee D S, Seo J M, Ahn J K, Yu Y S, Lee W J, et al. The number of CD8+ T cells and NKT cells increases in the aqueous humor of patients with Behcet's uveitis. Clinical and experimental immunology. 2004; 137(2):437-43.
Zang Y C, Li S, Rivera V M, Hong J, Robinson R R, Breitbach W T, et al. Increased CD8+ cytotoxic T cell responses to myelin basic protein in multiple sclerosis. Journal of immunology. 2004; 172(8):5120-7.

1. A method of treating a subject comprising administering to a subject in need thereof an effective amount of a killer cell lectin-like receptor G1 (KLRG1) depleting agent, thereby depleting CD8+ cytotoxic T and/or NK cells in vivo.

2. A method of claim 1, wherein the KLRG1 depeleting agent has effector killing function.

3. The method of claim 1, wherein the KLRG1 depleting agent is an antibody or antigen binding fragment thereof, or antibody mimetic.

4. The method of claim 3, wherein the antibody is monoclonal.

5. The method of claim 3, wherein the antibody or antigen binding fragment thereof, or antibody mimetic comprises a human or humanized antibody.

6. The method of claim 3, wherein the antibody or antigen binding fragment thereof, or antibody mimetic comprises:
a. a full length antibody Fab antibody that binds KLRG1 with effector function antibody dependent cell-mediated cytotoxicity (ADCC);
b. an antibody that binds KLRG1 with effector function complement dependent cytotoxicity (CDC);
c. an antibody that binds KLRG1 with effector function antibody-drug conjugate (ADC);
d. an Fc-cadherin fusion protein;
e. a fusion protein E-cadherin/Fc;
f. a fusion protein R-cadherin/Fc;
g. a fusion protein N-cadherin/Fc;
h. a chimeric antigen receptor; or
i. a multispecific antibody.

7. The method of claim 6, comprising the chimeric antigen receptor and wherein the chimeric antigen receptor comprises a specificity portion of a KLRG1 antibody grafted onto a T cell.

8. The method of claim 6, comprising a multispecific antibody and wherein the multispecific antibody comprises a bispecific or trispecific antibody.

9. The method of any one of claims 1-8, wherein the depleting agent binds KLRG1 extracellular domain.

10. The method of claim 9, wherein the KLRG1 is the extracellular domain of human KLRG1 isotype 1 or 2.

11. The method of claim 9 or 10, wherein the depleting agent cross reacts with the extracellular domains of human and cynomolgus KLRG1.

12. The method of claim 9 or 10, wherein the depleting agent binds to an epitope of the extracellular domain of KLRG1, wherein the epitope is at least 90% identical in human and cynomolgus.

13. The method of claim 9 or 10, wherein the depleting agent binds to KLRG1 and is not (a) clone 13F12F2, 14C2A07, REA261, 13A2, SA231A2, 2F1, 13A2, or REA261; and/or (b) an agent described in PCT Application No. PCT/US17/35621.

14. The method of claim 9 or 10, wherein the depleting agent binds to KLRG1 and is not a mouse antibody.

15. The method of claim 9 or 10, wherein the depleting agent binds KLRG1, thereby labeling CD8+ cytotoxic T and/or NK cells for depletion.

16. The method of claim 9 or 10, wherein the depleting agent binds KLRG1, and induces Antibody-Dependent Cellular Cytotoxicity (ADCC) or Complement Dependent Cytotoxicity CDC.

17. The method of any one of claims 1-16, wherein the depleting agent selectively targets and depletes T and/or NK cells expressing KLRG1.

18. The method of any one of claims 1-17, wherein the depleting agent is administered by providing an mRNA encoding the depleting agent to the subject.

19. The method of any one of claims 1-18, wherein the subject has an autoimmune disease.

20. The method of claim 19, wherein the autoimmune disease is rheumatoid arthritis, psoriasis, inclusion body myositis (IBM), multiple sclerosis, ulcerative colitis, lymphocytic colitis, idiopathic thrombocytopenic purpura, or type 1 diabetes.

21. The method of any one of claims 1-18, wherein the subject has or is at risk of developing transplant rejection.

22. The method of claim 21, wherein the transplant rejection is kidney rejection.

23. The method of claim 24, wherein the kidney rejection is T cell mediated kidney rejection after transplantation.

24. The method of any one of claims 1-18, wherein the subject has a hematologic malignancy.

25. The method of claim 24, wherein the hematologic malignancy is a leukemia.

26. The method of claim 25, wherein the leukemia is T cell leukemia, NK cell leukemia, large granular lymphocytic leukemia (LGLL), or chronic lymphocytic leukemia (CLL).

27. The method of any one of claims 1-18, wherein the subject has a lymphoma.

28. The method of claim 27, wherein the lymphoma is a T cell lymphoma, preferably anaplastic large cell lymphoma.

29. The method of any one of claims 1-18, wherein the subject has a solid tumor.

30. The method of claim 29, wherein the solid tumor is a breast cancer, gastric cancer, ovarian cancer, prostate cancer, glioma, glioblastoma, melanoma, lung cancer, kidney cancer, or tongue cancer.

31. A killer cell lectin-like receptor G1 (KLRG1) depleting agent for use in cell depletion, optionally excluding any of the agents described in PCT Application No. PCT/US17/35621.

32. The depleting agent of claim 31, wherein the depleting agent is an antibody or antigen binding fragment thereof, or antibody mimetic comprises:
a. a full length antibody Fab antibody that binds KLRG1 with effector function antibody dependent cell-mediated cytotoxicity (ADCC);
b. an antibody that binds KLRG1 with effector function complement dependent cytotoxicity (CDC);
c. an antibody that binds KLRG1 with effector function antibody-drug conjugate (ADC);
d. an Fc-cadherin fusion protein;
e. a fusion protein E-cadherin/Fc;
f. a fusion protein R-cadherin/Fc;
g. a fusion protein N-cadherin/Fc;
h. a chimeric antigen receptor; or
i. a multispecific antibody.

33. An mRNA or cDNA encoding the depleting agent of claim 31 or 32.

34. A pharmaceutical composition for use as the depleting agent in the method according to any of claims 1-18.

35. The method of any one of claims 24-30, further comprising administering to the subject an effective amount of a checkpoint modulator therapy.

36. The method of claim 35, wherein the KLRG1 depleting agent and checkpoint modulator therapy are synergistic.

37. The method of any one of claims 35 or 36, wherein the checkpoint modulator therapy comprises an anti-PD-1, anti-PD-L1, or anti-CTLA-4 therapy.

38. The method of any of claims 24-30 or 35-37, wherein the subject has failed or has not responded to a prior cancer therapy.
